(19) United States
(12) Patent Application Publication
(10) Pub. No.: US 2024/0293471 A1
(43) Pub. Date: Sep. 5, 2024

(54) PLACENTAL CELL TREATMENT FOR CRITICAL LIMB ISCHEMIA PATIENT SUBPOPULATIONS

(71) Applicant: PLURI BIOTECH LTD., Haifa (IL)

(72) Inventors: Daniel ROTHENSTEIN, Ramat Hasharon (IL); Yuval MATHEWS, Tel-Aviv (IL)

(21) Appl. No.: 18/689,276

(22) PCT Filed: Aug. 29, 2022
(86) PCT No.: PCT/IL2022/050937

§ 371 Date: Mar. 5, 2024

Related U.S. Application Data
(60) Provisional application No. 63/242,087, filed on Sep. 9, 2021.
(60) Provisional application No. 63/251,702, filed on Oct. 4, 2021.
(60) Provisional application No. 63/283,615, filed on Nov. 29, 2021.

(51) Int. Cl. A61K 35/50; A61K 9/00; A61P 9/10; C12N 5/073; G01N 33/66; G01N 33/68

(52) U.S. Cl. CPC A61K 35/50; A61K 9/0019; A61P 9/10; C12N 5/0605; G01N 33/66; G01N 33/6893; G01N 2333/805; G01N 2800/042

(57) ABSTRACT

Disclosed herein are methods and compositions comprising placental adherent stromal cells for treating peripheral ischemic disease, for example critical limb ischemia in specific patient subpopulations; and methods of selecting subjects having peripheral ischemic disease, for example critical limb ischemia, amenable for treatment with placental adherent stromal cells.

### FIELD

Disclosed herein are methods and compositions for treating critical limb ischemia using placental adherent stromal cells.

### BACKGROUND

Placental adherent stromal cells are known for use in treating peripheral artery disease such as critical limb ischemia (CLI) (WO 2009/037690 to Amir Toren et al.), the end-stage of peripheral artery disease (PAD). CLI is caused by tissue hypoxia and characterized by ischemic rest pain, ulcers, or gangrene associated with a significant risk of affected limb loss and a high risk for cardiovascular events. The annual incidence is approximately 500-1000 new cases per million in industrialized countries. Current treatment options are based on endovascular intervention, bypass surgery, and infusion of vasoactive agents as a potential adjunct therapy. Over the past two decades, many therapeutic advances have been accomplished in the field of PAD revascularization techniques. Modern technologies improve short-term outcomes of interventions, though they fail to improve the long-term prognosis. Moreover, although surgical or endovascular revascularization improves macrovascular perfusion, microvascular perfusion often remains unimproved. Furthermore, despite all medical advancements, 20-45% of patients are unsuitable for a revascularization procedure. This subgroup of patients is burdened with a high risk of limb loss, increased morbidity, and mortality (Jaluvka F et al.) Thus, there is a critical need to develop novel therapeutic strategies to improve limb perfusion and healing process, both for patients unsuitable for revascularization and as adjunct therapy for patients undergoing revascularization.

Cell therapy has been proposed as treatment for CLI. Yet despite some indications of promise and colossal expenditures of time, effort and financial resources on clinical trials exceeding 1500 patients (Jaluvka F et al.), not a single pivotal trial has been successfully concluded to date.

### SUMMARY

In a first aspect, the present invention provides a method for treating peripheral ischemia in a subpopulation of peripheral ischemia subjects, comprising selecting a peripheral ischemia subject wherein the subject has a normal glycemic status; and administering a therapeutically effective amount of a population of placental adherent stromal cells (ASC), therefore treating peripheral ischemia.

In another aspect, the present invention provides a method for treating critical limb ischemia (CLI) or increasing amputation-free survival (AFS) in a subpopulation of CLI subjects, comprising selecting a CLI subject wherein the subject has a normal glycemic status; and administering a therapeutically effective amount of a population of placental adherent stromal cells (ASC), therefore treating CLI or increasing amputation-free survival (AFS).

In another aspect, the present invention provides a method of treating critical limb ischemia (CLI) in a patient, the method comprising the steps of:
(a) determining whether the patient has diabetes mellitus; and
(b) if the patient does not have diabetes mellitus, then intramuscularly administering placental ASC to the patient, and
(c) if the patient does have diabetes mellitus, then administering an alternative therapy to the patient.

In certain embodiments, said treating CLI comprises one or more of increasing AFS, decreasing likelihood of requiring revascularization, decreasing likelihood of developing gangrene, inhibiting development of necrosis, healing wounds, decreasing likelihood of worsening of wounds, or improving perfusion of an ischemic limb.

In another aspect, the present invention provides a method of increasing amputation-free survival (AFS) in a subject with critical limb ischemia (CLI), comprising: (a) screening the subject for the presence or absence of diabetes mellitus; (b) administering placental adherent stromal cells (ASC) to the subject if said diabetes mellitus is determined to be absent; and (c) applying an alternative therapy to the subject if said diabetes mellitus is determined to be present, thereby increasing AFS in a subject with CLI.

In one embodiment, said step of determining whether the patient has diabetes mellitus is performed by:
(i) obtaining or having obtained a biological sample(s) from the patient; and
(ii) performing or having performed an assay on the biological sample to measure a parameter selected from the group consisting of hemoglobin A1C glycosylation, post-prandial plasma glucose level, and fasting plasma glucose level.

In another aspect, the present invention provides a composition for treating peripheral ischemia, comprising placental adherent stromal cells (ASC), wherein said composition is indicated for use in a subject with normal glycemic status.

In another aspect, the present invention provides a composition for treating critical limb ischemia (CLI), said composition comprising placental adherent stromal cells (ASC), wherein said composition is indicated for use in a subject with normal glycemic status.

In one embodiment, said treating CLI comprises one or more of increasing AFS, decreasing likelihood of requiring revascularization, decreasing likelihood of developing gangrene, inhibiting development of necrosis, healing wounds, decreasing likelihood of worsening of wounds, or improving perfusion of an ischemic limb.

In another aspect, the present invention provides a composition for increasing amputation-free survival (AFS) in a subject with critical limb ischemia (CLI), said composition comprising placental adherent stromal cells (ASC), wherein said composition is indicated for use in a subject with normal glycemic status.

In one embodiment, said composition is an injected composition.

In one embodiment, said placental ASC have been incubated on a 2D substrate.

In another embodiment, said placental ASC have been incubated on a 3D substrate.

In yet another embodiment, said placental ASC have been incubated on a 2D substrate, prior to incubating on a 3D substrate.

In one embodiment, said 3D culture substrate comprises a synthetic adherent material.

In one embodiment, said synthetic adherent material is a fibrous matrix.

In certain embodiments, said synthetic adherent material is selected from the group consisting of a polyester, a polypropylene, a polyalkylene, a polyfluorochloroethylene, a polyvinyl chloride, a polystyrene, and a polysulfone.

In one embodiment, said 3D culture apparatus comprises microcarriers disposed within a bioreactor.

In one embodiment, said placental ASC are allogeneic to said subject.

In one embodiment, the composition is intramuscularly injected.

In certain embodiments said composition comprises, or said method comprises the administration of, 100-600 million of said placental ASC, for an adult subject.

In one embodiment, said placental ASC express a marker selected from the group consisting of CD73, CD90, CD29 and CD105.

In one embodiment, said placental ASC do not express a marker selected from the group consisting of CD3, CD4, CD11b, CD14, CD19, and CD34.

In one embodiment, said placental ASC do not express a marker selected from the group consisting of CD3, CD4, CD11b, CD14, CD19, and CD34.

In one embodiment, said placental ASC do not express a marker selected from the group consisting of CD3, CD4, CD34, CD39, and CD106.

In one embodiment, less than 50% of said placental ASC express CD200.

In another embodiment, more than 50% of said placental ASC express CD200.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the embodiments of the invention only and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a diagram of a bioreactor that can be used to prepare the cells.

FIG. 2A-J contain pictures of bone marrow (BM)-derived mesenchymal stem cells (MSC) (FIGS. 2A and 2F) or placental cells after adipogenesis assays (FIGS. 2B-2E and 2G-2J). Cells were incubated with (FIG. 2A-2E) or without (FIG. 2F-2J) differentiation medium. Placental ASCs were expanded in serum replacement medium (SRM) (FIG. 2B, 2G; FIG. 2C, 2H; and FIG. 2D, 2I depicting 3 different batches) or in full DMEM (FIG. 2E, 2J). Arrows indicate cells stained positively with Oil Red O.

FIG. 3A-J contain pictures of BM-derived MSC (FIGS. 3A and 3F) or placental cells after osteogenesis assays (FIGS. 3B-3E and 3G-3J). Cells were incubated with (FIG. 3A-3E) or without ((FIG. 3F-3J) differentiation medium. Placental ASCs were expanded in SRM (FIG. 3B, 3G; FIG. 3C, 3H; and FIG. 3D, 3I depicting 3 different batches) or in full DMEM (FIG. 3E, 3J). Arrows indicate cells stained positively with Alizarin Red S.

FIG. 4 is a Venn Diagram of selected risk factors in the clinical study.

FIG. 5 is a Kaplan Meier curve of product-limit survival estimates through the duration of the study, with number of patients at risk. Solid line: ASC-treated; Dotted line: placebo treated. Plotted is percent amputation-free survival (primary endpoint; vertical axis) vs. days elapsed since randomization (horizontal axis).

FIG. 6 is a plot showing percentage (vertical axis) of subjects in the diabetic cohort experiencing death or major amputation in ASC-treated (solid line) or vehicle-treated (dotted line) subjects.

FIG. 7 is a hazard ratio plot of primary endpoint subgroup analysis.

FIG. 8 is a Kaplan Meier curve of product-limit survival estimates through the first 400 days, in the non-diabetic cohort. Solid line: ASC-treated. Dotted line: placebo. Plotted is percent amputation-free survival (primary endpoint; vertical axis) vs. days elapsed since randomization (horizontal axis).

FIG. 9 A-F are graphs showing ASC in vitro activity in normal and hyperglycemic conditions. Vertical axis: apoptosis (A), viability (B), relative potency as measured in a cell adhesion assay (CAA; C), cell numbers representing population doubling time (PDT) (D), VEGF secretion (E), and endothelial cell proliferation (ECP) (F). Horizontal axis: glucose concentration in millimoles (mM) (A, B, C, and E); days (D); or treatment groups: + or −ASC CM (with or without adherent placental cells conditioned medium), HD (healthy donor), T2D (type 2 diabetic patients) (F). For (D), data points glucose concentrations of 5, 25, and 50 mM are shown as diamonds, squares, and triangles, respectively. Bar graphs (A, B, C, E, and F) depict Mean±SD of the values for each assay. For (F) asterisks show the significance between individual pairs as indicated, *p<0.05, **p<0.01, ***p<0.001, ****p<0.0001. One-way ANOVA, followed by Tukey's multiple comparisons test.

FIG. 10A-B are graphs depicting percentage of blood flow (vertical axis) at various timepoints (horizontal axis shows number of days) following artery ligation and treatment with ASC or vehicle (placebo). In (A), data from wt (C57/BL) mice vs. db/db mice are shown as black and gray lines, respectively. Placebo-treated mice and low- and high-dose ASC-treated mice are shown as dots, dashes, and solid lines, respectively. (B) shows only high-dose mouse data. Data points and error bars depict Mean±SD of the values.

FIG. 11 is a graph depicting CD34 staining (% area average±SEM) for each of the treated groups: 1M (PlasmaLyte) db/db, 3M (High dose) db/db, 4M (PlasmaLyte) C57B1, and 6M (High dose) C57B1.

### DETAILED DESCRIPTION

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Aspects of the invention relate to methods and compositions that comprise placental adherent stromal cells (ASC) and their conditioned media (CM). In some embodiments, the ASC may be human ASC, or in other embodiments animal ASC.

In one embodiment, there is provided a method for treating critical limb ischemia (CLI) in a subpopulation of CLI subjects, comprising: selecting a CLI subject for treatment based on the subject's normal glycemic status (i.e., selecting a CLI subject wherein the subject has a normal glycemic status); and administering a therapeutically effective amount of a population of placental ASC to the subject, therefore treating CLI. In certain embodiments, the subject is a human.

The terms “patient” and “subject” are used interchangeably herein.

In another embodiment, there is provided a method for treating peripheral ischemia, for example intermittent claudication (IC), in a subpopulation of subjects, comprising: selecting an ischemic subject for treatment based on the subject's normal glycemic status; and administering a therapeutically effective amount of a population of placental ASC to the subject, therefore treating peripheral ischemia, e.g., IC. In certain embodiments, the subject is a human.

In another embodiment, there is provided a method of decreasing an incidence of requiring amputation in a subpopulation of CLI subjects—or, in other embodiments, in a population of CLI subjects, or increasing amputation-free survival (AFS) in a subpopulation of CLI subjects comprising: selecting a CLI subject for treatment based on the subject's normal glycemic status; and administering a therapeutically effective amount of a population of placental ASC to the subject, therefore decreasing an incidence of requiring amputation. In certain embodiments, the subject is a human.

In yet another embodiment, there is provided a method of decreasing an incidence of requiring revascularization (or repeat revascularization) in a subpopulation of CLI subjects—or, in other embodiments, in a population of CLI subjects, comprising: selecting a CLI subject for treatment based on the subject's normal glycemic status; and administering a therapeutically effective amount of a population of placental ASC to the subject, therefore decreasing an incidence of requiring revascularization. In certain embodiments, the subject is a human.

In various embodiments, placental ASC are administered to the subject within 1, 2, 3, 4, 7, 10, 14, 21, 30, 45, 60, 90, 120, or 180 days after a first revascularization.

In yet another embodiment, there is provided a method of decreasing an incidence of gangrene in a subpopulation of CLI subjects—or, in other embodiments, in a population of CLI subjects, comprising: selecting a CLI subject for treatment based on the subject's normal glycemic status; and administering a therapeutically effective amount of a population of placental ASC to the subject, therefore decreasing an incidence of gangrene. In certain embodiments, the subject is a human.

Placental ASC alter cytokine secretion by PBMC and stimulate ECP (endothelial cell proliferation) (PCT/IL2021/050268 to Yaacob Yanay et al.). As provided herein, CLI subjects not exhibiting diabetes mellitus (e.g., with Hemoglobin A1A [HbA1C]<6.4%) exhibited a significant positive response to treatment with placental ASC. It is hereby demonstrated that non-diabetic subjects are particularly suited to treatment with placental ASC. Interestingly, although placental ASC retain their pro-angiogenic activity under high glucose conditions, diabetic subjects exhibit lower responsiveness to treatment of ischemia with the ASC.

In other embodiments, there is a provided a method of treating a subject for CLI. In other embodiments, there is a provided a method of benefitting a subject with CLI. In certain embodiments, the benefit is decreasing likelihood of requiring amputation, or of requiring revascularization, or of developing gangrene; inhibiting development of necrosis, healing wounds, decreasing likelihood of worsening of wounds, and/or improving perfusion of an ischemic limb, each of which represents a separate embodiment. In one embodiment, the method comprises administering an effective dose of placental ASC to the subject, optionally after ascertaining the subject's glycemic status. In some embodiments, a subject exhibits a normal glycemic status. The normal glycemic status can predict sensitivity to treatment with placental ASC. In some embodiments, an alternative therapy is administered to the subject, if an abnormal glycemic status predicts resistance to treatment with placental ASC.

In other embodiments, there is a provided a method for identifying a human subject with CLI likely to benefit from treatment with placental ASC and treating the subject accordingly, with the method comprising: (a) determining a diabetes mellitus status of the subject, by use of methods including measurement of hemoglobin A1C glycosylation, post-prandial plasma glucose level, and fasting plasma glucose level; and (b) if the subject is identified as non-diabetic, treating the patient with placental ASC, therefore treating CLI. In certain embodiments, the diabetes mellitus status of the subject is determined by one or more methods selected from measurement of hemoglobin A1C glycosylation, post-prandial plasma glucose level, and fasting plasma glucose level. In other embodiments, treatment of CLI confers a benefit selected from the benefits mentioned herein, each of which represents a separate embodiment.

In other embodiments, there is a provided a method of treating a subject for CI. In other embodiments, there is a provided a method of benefitting a subject with CI. In certain embodiments, the benefit is improved prognosis. In one embodiment, the method comprises administering an effective dose of placental ASC to the subject, optionally after ascertaining the subject's glycemic status. In some embodiments, a subject exhibits a normal glycemic status. The normal glycemic status can predict sensitivity to treatment with placental ASC. In some embodiments, an alternative therapy is administered to the subject, if an abnormal glycemic status predicts resistance to treatment with placental ASC.

In other embodiments, there is a provided a method for identifying a human subject with CI likely to benefit from treatment with placental ASC and treating the subject accordingly, with the method comprising: (a) determining a diabetes mellitus status of the subject, by use of methods including measurement of hemoglobin A1C glycosylation, post-prandial plasma glucose level, and fasting plasma glucose level; and (b) if the subject is identified as non-diabetic, treating the patient with placental ASC, therefore treating CI. In certain embodiments, the diabetes mellitus status of the subject is determined by one or more methods selected from measurement of hemoglobin A1C glycosylation, post-prandial plasma glucose level, and fasting plasma glucose level. In other embodiments, treatment of CI confers a benefit selected from the benefits mentioned herein, each of which represents a separate embodiment.

In still other embodiments, there is provided a method of treating a patient with placental ASC or an alternative therapy, wherein the patient is suffering from CLI, the method comprising the steps of:
(a) determining whether the patient has diabetes mellitus by: (i) obtaining or having obtained a biological sample(s) from the patient; and (ii) performing or having performed an assay on the biological sample to measure a parameter selected from the group consisting of hemoglobin A1C glycosylation, post-prandial plasma glucose level, and fasting plasma glucose level; in order to determine if the patient has diabetes mellitus; and
(b) if the patient does not have diabetes mellitus, then intramuscularly administering placental ASC to the patient, and
(c) if the patient does have diabetes mellitus, then administering an alternative therapy to the patient,
wherein a likelihood of successful treatment of CLI with placental ASC is higher for a patient not having diabetes mellitus than it would be for a patient having diabetes mellitus.

In yet other embodiments, there is a provided a method of treating a subject having CLI, comprising: (a) screening the subject for the presence or absence of diabetes mellitus; (b) administering placental ASC to the subject if the diabetes mellitus is determined to be absent; and (c) applying an alternative therapy to the subject if the diabetes mellitus is determined to be present, thereby treating the CLI. In certain embodiments, the benefit of treatment is any of the benefits mentioned herein, each of which represents a separate embodiment.

In yet another aspect, the disclosure provides a method of treating CLI with placental ASC. In one embodiment, the method comprises (a) assessing an indicator of glycemic status in a biological sample; and (b) administering an effective dose of the placental ASC to the subject if the indicator indicates normal glycemic status as compared to a control sample and/or a reference level. The normal glycemic status may be a predictor of placental ASC sensitivity. In some embodiments, the glycemic status is assessed by hemoglobin A1C glycosylation, post-prandial plasma glucose level, or fasting plasma glucose level. In certain embodiments, the benefit of treatment is any of the benefits mentioned herein, each of which represents a separate embodiment. Alternatively, or in addition, the placental ASC are administered intramuscularly.

In still other embodiments, there is provided a method for increasing likelihood of amputation-free survival (AFS) in a subpopulation of CLI subjects, comprising: selecting a CLI subject for treatment based on the subject's normal glycemic status (e.g., with HbA1C<6.4%); and administering a therapeutically effective amount of a population of placental ASC, therefore increasing likelihood of AFS in a subpopulation of CLI subjects. As provided herein, CLI subjects not exhibiting diabetes mellitus exhibited a significant positive response to treatment with placental ASC.

In further embodiments, there is provided a method of increasing likelihood of AFS in a subject with CLI, comprising: (a) screening the subject for the presence or absence of diabetes mellitus; (b) administering placental ASC to the subject if the diabetes mellitus is determined to be absent; and (c) applying an alternative therapy to the subject if the diabetes mellitus is determined to be present, thereby increasing likelihood of AFS in a subject with CLI.

In certain embodiments, successful treatment of CLI can be assessed by a lack of deterioration in ambulatory status, lack of requirement to amputate the affected limb(s), wound closure, and/or quality of life, e.g., as assessed by the Vascular Quality of Life (VascuQoL) questionnaire (Alabi et al., deVries et al., and the references cited therein).

In other embodiments, there is a provided a composition for treating CLI, comprising placental ASC, wherein the composition is indicated for use in a subject with normal glycemic status.

In still other embodiments, there is provided a composition for increasing likelihood of AFS in a subject with CLI, comprising placental ASC, wherein the composition is indicated for use in a subject with normal glycemic status.

In still other embodiments, there is provided a composition for decreasing likelihood of a subject with CLI requiring amputation, comprising placental ASC, wherein the composition is indicated for use in a subject with normal glycemic status.

In still other embodiments, there is provided a composition for decreasing likelihood of a subject with CLI requiring revascularization (or, in some embodiments, repeat revascularization), comprising placental ASC, wherein the composition is indicated for use in a subject with normal glycemic status.

In still other embodiments, there is provided a composition for decreasing likelihood of a subject with CLI developing gangrene, comprising placental ASC, wherein the composition is indicated for use in a subject with normal glycemic status.

In still other embodiments, there is provided a composition for inhibiting development of necrosis in a subject with CLI, comprising placental ASC, wherein the composition is indicated for use in a subject with normal glycemic status.

In still other embodiments, there is provided a composition for healing wounds (e.g., ischemic wounds) of a subject with CLI, comprising placental ASC, wherein the composition is indicated for use in a subject with normal glycemic status.

In still other embodiments, there is provided a composition for decreasing likelihood of worsening of wounds (e.g., ischemic wounds) of a subject with CLI, comprising placental ASC, wherein the composition is indicated for use in a subject with normal glycemic status.

In still other embodiments, there is provided a composition for improving perfusion of an ischemic limb(s) in a subject afflicted with CLI, comprising placental ASC, wherein the composition is indicated for use in a subject with normal glycemic status. In certain embodiments, the composition is indicated for intramuscular administration in the ischemia limb(s).

CLI, as used herein, refers to a condition characterized by chronic ischemic at-rest pain, ulcers, and/or gangrene in one or both legs attributable to objectively proven arterial occlusive disease. In certain embodiments, CLI implies chronicity and is to be distinguished from acute limb ischemia. In some embodiments, the treated patient(s) exhibit(s) atherosclerotic CLI with minor tissue loss up to the ankle level. In certain embodiments, the patient(s) is/are Rutherford Category 5. In other embodiments, the patient(s) exhibit(s) nonhealing ulcer(s) and/or focal gangrene with diffuse pedal ischemia. In certain embodiments, a Rutherford Category 5 patient(s) exhibit(s) resting ankle pressure <60 mm Hg in the affected leg(s), ankle or metatarsal pulse volume recording flat or barely pulsatile, and/or toe pressure <40 mm Hg (Hardman et al.). Alternatively or in addition, the patient(s) is/are unsuitable for revascularization and/or carry/ies an unfavorable risk benefit for revascularization (Norgren et al.).

In yet other embodiments, there is provided a method of measuring a likelihood that a subject having CLI will exhibit a clinically beneficial response to treatment with placental ASC. In some embodiments, the method comprises (a) measuring, in a sample from the subject, glycemic indicators, for example hemoglobin A1C glycosylation, post-prandial plasma glucose level, and/or fasting plasma glucose level. In further embodiments, a method of the disclosure further comprises (b) generating a diabetic profile based on a comparison between the glycemic control indicators in the sample(s) from the subject and corresponding values in a control sample. In some embodiments, a method of the disclosure further comprises calculating, using a computer system and/or a computational algorithm, a likelihood of response of the subject to treatment with placental ASC based on the glycemic control indicators. In certain embodiments, the benefit of treatment is any of the benefits mentioned herein, each of which represents a separate embodiment.

In other embodiments, the disclosure provides a method of selecting a therapy against CLI. In one embodiment, the method comprises (a) obtaining a pretreatment sample from a subject; (b) measuring one or more glycemic control indicators in the pretreatment sample to determine the subject's diabetic profile relative to a control sample and/or a reference level; and (c) selecting placental ASC for treatment of the subject based on the subject's non-diabetic status. In some embodiments, non-diabetic status is a predictor of placental ASC sensitivity. If the diabetic profile (e.g., presence of diabetes) predicts a likelihood of resistance to treatment with placental ASC, an alternative therapy may be selected. In certain embodiments, the benefit of treatment is any of the benefits mentioned herein, each of which represents a separate embodiment.

In still other embodiments, the disclosure provides a method of categorizing a CLI status of a subject. In one embodiment, the method comprises (a) obtaining a sample from the subject; (b) measuring one or more glycemic control indicators in the sample; (c) generating a diabetic profile based on a comparison between glycemic control indicator values in the sample from the subject and corresponding values of a reference sample derived from a different subject having a known CLI status; and (d) categorizing the CLI status of the subject based on the indicator values. In some embodiments, a diabetic profile predicts for sensitivity or resistance to treatment with placental ASC. In certain embodiments, the benefit of treatment is any of the benefits mentioned herein, each of which represents a separate embodiment.

In yet other embodiments, a method of the disclosure comprises a biomarker that, if out of normal range, is a predictor of placental ASC resistance in a subject with CLI, the biomarker including, for example, hemoglobin A1C glycosylation, post-prandial plasma glucose level, and/or fasting plasma glucose level. In some embodiments, abnormal value(s) of the biomarker predict(s) resistance to placental ASC.

In certain embodiments, a method of the disclosure is particularly useful for evaluating whether placental ASC will have a desired effect, i.e., predicting responsiveness of CLI to placental ASC, and determining prognoses. The present methods may be used for the optimization of treatment protocols. In this context, evaluation of a subject's diabetic status can be used to gain information on the treatment potential of placental ASC. In certain embodiments, the benefit of treatment is any of the benefits mentioned herein, each of which represents a separate embodiment.

In some embodiments, glycemic control indicators are used in a method of the disclosure to assess a likelihood of response to treatment of CLI with placental ASC. The likelihood of response may be adjusted downward for each indicator value that is out of normal range. In some embodiments, the likelihood of response may be adjusted upward for each indicator value that is within normal range. In certain embodiments, the benefit of treatment is any of the benefits mentioned herein, each of which represents a separate embodiment.

In some embodiments, one or more steps in the assessment and/or reporting of a likelihood of response to treatment with placental ASC is performed with the aid of a processor, such as with a computer system executing instructions contained in computer-readable media. In one aspect, the disclosure provides a system for of assessing a likelihood of a subject having CLI exhibiting a clinically beneficial response to treatment with placental ASC. In one embodiment, the system comprises (a) a memory unit configured to store information concerning an expression level of a plurality of glycemic control indicators in a biological sample(s) from the subject, wherein the glycemic control indicators predict for sensitivity or resistance to treatment with placental ASC. In some embodiments, the system further comprises (b) one or more processors alone or in combination programmed to (1) determine a weighted probability of placental ASC responsiveness based on a plurality of glycemic control indicators as compared to corresponding values in one or more control samples and (2) designate the subject as having a high probability of exhibiting a clinically beneficial response to treatment with placental ASC if the weighted probability corresponds to at least 2 times a baseline probability, where the baseline probability represents a likelihood that the subject will exhibit a clinically beneficial response to treatment with placental ASC before obtaining the weighted probability of (b)(1). In some embodiments, a processor or computational algorithm may aid in the assessment of a likelihood of a subject having CLI exhibiting a clinically beneficial response to treatment with placental ASC. For example, one or more steps of methods or systems described herein may be implemented in hardware. Alternatively, one or more steps may be implemented in software stored in, for example, one or more memories or other computer readable medium and implemented on one or more processors. As is known, the processors may be associated with one or more controllers, calculation units, and/or other units of a computer system, or implanted in firmware as desired. If implemented in software, the routines may be stored in any computer readable memory such as in RAM, ROM, flash memory, a magnetic disk, a laser disk, a remote server (e.g., the cloud), or other storage medium, as is also known. Likewise, this software may be delivered to a computing device via any known delivery method including, for example, over a communication channel such as a telephone line, the internet, a wireless connection, etc., or via a transportable medium, such as a computer readable disk, flash drive, etc. The various steps may be implemented as various blocks, operations, tools, modules and techniques which, in turn, may be implemented in hardware, firmware, software, or any combination of hardware, firmware, and/or software. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc. A computer system may be involved in one or more of sample collection, sample processing, data analysis, expression profile assessment, calculation of weighted probabilities, calculation of baseline probabilities, comparison of a weighted probability to a reference level and/or control sample, determination of a subject's absolute or increased probability, generating a report, and reporting results to a receiver.

A client-server, relational database architecture can be used in embodiments of the disclosure. A client-server architecture is a network architecture in which each computer or process on the network is either a client or a server. Server computers are typically powerful computers dedicated to managing disk drives (file servers), printers (print servers), or network traffic (network servers). Client computers include PCs (personal computers), workstations, or mobile computing devices (e.g., a tablets or smart phones) on which users run applications, as well as example output devices as disclosed herein. Client computers may rely on server computers for resources, such as files, devices, and even processing power. In some embodiments of the disclosure, the server computer handles all of the database functionality. The client computer can have software that handles all the front-end data management and can also receive data input from users.

In some embodiments, the computer system is connected to an analysis system by a network connection. The computer system may be understood as a logical apparatus that can read instructions from media and/or a network port, which can optionally be connected to server having fixed media. The system can include a CPU, disk drives, optional input devices such as keyboard and/or mouse, and optional monitor. Data communication can be achieved through the indicated communication medium to a server at a local or a remote location. The communication medium can include any means of transmitting and/or receiving data. For example, the communication medium can be a network connection, a wireless connection, or an internet connection. Such a connection can provide for communication over the internet or World Wide Web. In some embodiments, a physical report is generated and delivered to a receiver.

In some embodiments there is provided a computer readable medium encoded with computer executable software that includes instructions for a computer to execute functions associated with the identified biomarkers. Such computer system may include any combination of such codes or computer executable software, depending upon the types of evaluations desired to be completed. The system can have code for calculating a weighted probability of placental ASC responsiveness, and optionally for calculating an aggregated probability based on a plurality of weighted probabilities. In some embodiments, the weighted probability of placental ASC responsiveness is increased if the subject exhibits one or more indications of healthy glucose metabolism or (2) does not exhibit one or more biomarkers of diabetes mellitus. The subject may express predictors of both sensitivity and resistance. In calculating a weighted probability, the computer system or computational algorithm may consider the expression of 1 or more, 2 or more, 3 or more, 4 or more, 5 or more, 6 or more, 7 or more, 8 or more, 9 or more, 10 or more, 15 or more, 20 or more, 25 or more, 30 or more, 40 or more, 50 or more, 60 or more, 70 or more, 80 or more, 90 or more, or 100 or more biomarkers. For example, expression levels of all of the biomarkers in Table 5 can be used to generate an expression profile. The system can further comprise code for conducting genetic analysis based on specific panel(s) of biomarkers chosen. The system can also have code for one or more of the following: conducting, analyzing, organizing, or reporting the results, as described herein. The system can also have code for generating a report. In some embodiments, the test subject may be designated as having a high probability of exhibiting a clinically beneficial response to treatment with placental ASC if the weighted probability corresponds to at least about 0.55, at least about 0.6, at least about 0.65, at least about 0.7, at least about 0.75, at least about 0.8, at least about 0.85, at least about 0.9, at least about 0.95, or at least about 0.99. In some embodiments, the test subject may be designated as having a low probability of exhibiting a clinically beneficial response to treatment with placental ASC if the weighted probability corresponds to less than about 0.45, less than about 0.4, less than about 0.35, less than about 0.3, less than about 0.25, less than about 0.2, less than about 0.15, less than about 0.1, less than about 0.05, or less than about 0.01.

The system may further comprise code for comparing a weighted probability to a baseline probability, a threshold value, and/or a reference level, and assigning a fold-baseline probability based on whether or not the baseline probability, threshold value, or reference level is exceeded. Assessing a weighted probability, threshold value, or reference level can be linked to at least one recommendation. Exceeding a weighted probability, threshold value, or reference level may be linked to a recommendation of treatment with placental ASC. In some embodiments, the baseline probability represents the average probability of a subject having CLI exhibiting a clinically beneficial response to treatment with placental ASC, either in general or for a specific population. In some embodiments, the baseline probability represents a pre-test likelihood that a particular subject will exhibit a clinically beneficial response to treatment with placental ASC before applying a method of the disclosure to determine a post-test risk. A weighted probability above a baseline probability may correspond to a specified fold-baseline probability, whatever the pre-test baseline for the subject may be. In some embodiments, the test subject may be designated as having a high probability of exhibiting a clinically beneficial response to treatment with placental ASC if the weighted probability corresponds to about, or in other embodiments at least about, 1.1-times, 1.2-times, 1.3-times, 1.4-times, 1.5-times, 1.8-times, 2-times, 2.5-times, 3-times, 4-times, 5-times, 6-times, 7-times, 8-times, 9-times, 10-times, 25-times, 50-times, or 100-times the baseline probability. In some embodiments, the test subject may be designated as having a low probability of exhibiting a clinically beneficial response to treatment with placental ASC if the weighted probability corresponds to about, or in other embodiments less than about, 0.9-times, 0.8-times, 0.7-times, 0.6-times, 0.5-times, 0.4-times, 0.3-times, 0.2-times, 0.1-times, 0.05-times, 0.01-times the baseline probability.

After performing a calculation, a processor can provide the output, such as from a calculation, back to, for example, the input device or storage unit, to another storage unit of the same or different computer system, or to an output device. Output from the processor can be displayed by data display. A data display can be a display screen (for example, a monitor or a screen on a digital device), a print-out, a data signal (for example, a packet), an alarm (for example, a flashing light or a sound), a graphical user interface (for example, a webpage), or a combination of any of the above. In an embodiment, an output is transmitted over a network (for example, a wireless network) to an output device. The output device can be used by a user to receive the output from the data-processing computer system. After an output has been received by a user, the user can determine a course of action, or can carry out a course of action, such as a medical treatment when the user is medical personnel. In some embodiments, an output device is the same device as the input device. Example output devices include, but are not limited to, a telephone, a wireless telephone, a mobile phone, a PDA, a tablet, a flash memory drive, a light source, a sound generator, a fax machine, a computer, a computer monitor, a printer, an iPod, and a webpage. The user station may be in communication with a printer or a display monitor to output the information processed by the server.

It is envisioned that data relating to the present disclosure can be transmitted over a network or connections for reception and/or review by a receiver. The receiver can be but is not limited to an individual; the subject to whom the report pertains; a health care provider, manager, other healthcare professional, or other caretaker; a vascular surgeon; a person or entity that performed and/or ordered the biomarker expression analysis; or a local or remote system for storing such reports (e.g., servers or other systems of a “cloud computing” architecture). In one embodiment, a computer-readable medium includes a medium suitable for transmission of a result of an analysis of a biological sample, such as analysis of one or more biomarkers. The medium can include a result regarding one or more biomarker expression levels of an individual, probability (such as fold-baseline probability) of the individual's CLI being sensitive to treatment with placental ASC, and/or a treatment plan for the individual, wherein such a result is derived using the methods described herein.

In some embodiments, the subject or a third party (e.g., a health care provider, health care manager, other health professional, or other caretaker) is alerted if a subject is designated as having a “high probability” of having a beneficial response to treatment with placental ASC. The analysis generated can be reviewed and further analyzed by a medical professional such as a managing doctor or licensed physician, or other third party. The medical professional or other third party can meet with the subject to discuss the results, analysis, and report. Information provided can include recommendations, such as treatment (e.g., with placental ASC or an alternative therapy).

In some embodiments, the method further comprises providing a recommendation for treatment based on an assessment of the likelihood that a subject having CLI will exhibit a clinically beneficial response to treatment with placental ASC, such as designation as having high probability. A recommendation may form part of a report generated based on biomarker expression analysis, or may be made by a receiver on the basis of such report. A recommendation may be for further action on the part of the subject and/or for a third party, such as a health care provider, health care manager, other health professional, or other caretaker. Recommendations may include, but are not limited to, treatment with placental ASC; continued monitoring of the subject; screening exams or laboratory tests that may further characterize the CLI; revascularization; and/or prescription or administration of one or more therapeutic agents that are not placental ASCs. In certain embodiments, the benefit of treatment is any of the benefits mentioned herein, each of which represents a separate embodiment.

In some embodiments, the disclosure provides a method of categorizing a CLI status of a subject. The CLI status of the subject may be categorized based on a sample from the subject. A CLI status may be categorized as likely sensitive to treatment with placental ASC or likely resistant to treatment with placental ASC. A “likely sensitive” categorization may be assigned to CLI having indicators of healthy glucose metabolism. A “likely resistant” categorization may be assigned to CLI having indicators of diabetes mellitus. A subject may have an expression profile having predictors of both sensitivity and resistance. In some embodiments, a subject may be categorized as sensitive if more biomarkers predict for sensitivity to placental ASC treatment than predict for resistance to placental ASC treatment.

In some embodiments, a method of the disclosure provides applying an alternative therapy to the subject if the subject is determined to have diabetes mellitus. The alternative therapy may comprise any treatment besides administration of placental ASC, for example, open surgical revascularization, endovascular revascularization, and antiplatelet and/or anticoagulant therapy.

### Compositions Comprising Conditioned Medium (CM)

Conditioned media/conditioned medium (CM), as used herein, refers to a growth medium that has been used to incubate a cell culture. The present disclosure is not intended to be limited to specific medium formulations; rather, any medium suitable for incubation of placental ASC is encompassed. Any of the embodiments described herein for ASC and methods of their incubation and expansion may also be applied to CM generated via the described ASC/methods.

In still another embodiment, there is a provided a method for treating a herein-described indication, utilizing a composition comprising CM derived from a cultured placental ASC. In various embodiments, the placental ASC are maternal tissue-derived ASC (ASC from a maternal portion of the placenta); fetal tissue-derived ASC (ASC from a fetal portion of the placenta); or a mixture thereof. Alternatively, or in addition, the placental ASC are allogeneic to the subject; or, in other embodiments, are autologous; or, in other embodiments, are xenogeneic. In certain embodiments, the composition is an injected composition, e.g., intramuscularly injected. In other embodiments, the composition is administered by another other route mentioned herein, each of which represents a separate embodiment.

### Compositions Comprising Exosomes

In still another embodiment, there is a provided a method for treating a herein-described indication, utilizing a composition comprising exosomes or other extracellular vesicles derived from a cultured placental ASC. In various embodiments, the placental ASC are maternal tissue-derived ASC (ASC from a maternal portion of the placenta); fetal tissue-derived ASC (ASC from a fetal portion of the placenta); or a mixture thereof. Alternatively, or in addition, the placental ASC are allogeneic to the subject; or, in other embodiments, are autologous; or, in other embodiments, are xenogeneic. In certain embodiments, the composition is an injected composition, e.g., intramuscularly injected. In other embodiments, the composition is administered by another other route mentioned herein, each of which represents a separate embodiment.

Methods of identifying and diagnosing diabetes mellitus are known in the art, and are described, for example, in Bergman M et al. In certain embodiments, diabetes mellitus is characterized by HbA1C level e.g., HbA1C>6.4%; or, in other embodiments, >6.5%; or, in other embodiments, >6.6%, >6.3%, >6.2%,>6.7%,>6.8%, or >6.9%. In yet other embodiments, diabetes is assessed by a competent physician, e.g., an endocrinologist. Those skilled in the art will appreciate that HbA1c increases with age independent of glucose tolerance (Davidson and Schriger) and is affected by ethnicity (Booth R A et al.) and genetic factors (Wheeler E, et al.).

In other embodiments, diabetes mellitus is diagnosed based on a fasting plasma glucose (FPG) concentration >126 mg/dl (7.8 mmol/L); or, in other embodiments, >140 mg/dl; or, in other embodiments, >135 mg/dl, >130 mg/dl, or >145 mg/dl.

In other embodiments, diabetes mellitus is diagnosed based on 2-h plasma glucose in the 75 g oral glucose tolerance test (2 hPG); e.g., in subjects with a 2-h PG level >240 mg/dl (13.33 mmol/L); or, in other embodiments, >200 mg/dl (11.11 mmol/L); or, in other embodiments, >210 mg/dl, >220 mg/dl, >230 mg/dl, or >250 mg/dl.

In another embodiment, there is provided use of ASC for the manufacture of a medicament for treating CLI, in a subject with normal glycemic status. In certain embodiments, the benefit(s) of treatment is/are any of the benefits mentioned herein or any combination thereof, each of which represents a separate embodiment.

In still other embodiments, there is provided an article of manufacture, comprising (a) a packaging material, wherein the packaging material comprises a label for use in any of the diseases, disorders, and complications mentioned herein, each of which represents a separate embodiment, specifying treatment of a non-diabetic subject; and (b) a pharmaceutical composition comprising placental ASC. In some embodiments, the pharmaceutical composition is frozen. In other embodiments, the label indicates use in treating CLI, decreasing likelihood of amputation or revascularization or gangrene, reducing necrosis, healing wounds, decreasing likelihood of worsening of wounds, and/or improving perfusion of an ischemic limb. As provided herein, administration of a therapeutically effective amount of placental ASC may be particularly useful in the aforementioned indications, in non-diabetic subjects.

In various embodiments, the described placental ASC are maternal tissue-derived ASC; fetal tissue-derived ASC; or a mixture thereof. Alternatively, or in addition, the placental ASC are allogeneic to the subject; or, in other embodiments, are autologous; or, in other embodiments, are xenogeneic. In other embodiments, conditioned medium (CM) of placental ASC is utilized in place of ASC. In other embodiments, a preparation of exosomes of placental ASC is utilized in place of ASC. In other embodiments, the composition is an injected composition, e.g., intramuscularly injected. Any of these embodiments may be freely combined with any of the therapeutic embodiments mentioned herein.

In still other embodiments, there is provided a composition for treating or ameliorating any of the diseases, disorders, and complications mentioned herein, each of which represents a separate embodiment, comprising cultured placental ASC. In other embodiments, the composition comprises placental ASC conditioned medium (“ASC-CM”).

### Basal Media for Expansion of ASC

Those skilled in the art will appreciate that growth media are utilized to expand the described placental ASC and/or produce the described CM for the compositions and methods described herein. Non-limiting examples of base media useful in 2D and 3D culturing include Minimum Essential Medium Eagle, ADC-1, LPM (Bovine Serum Albumin-free), F10(HAM), F12 (HAM), DCCM1, DCCM2, RPMI 1640, BGJ Medium (with and without Fitton-Jackson Modification), Basal Medium Eagle (BME—with the addition of Earle's salt base), Dulbecco's Modified Eagle Medium (DMEM—without serum), Yamane, IMEM-20, Glasgow Modification Eagle Medium (GMEM), Leibovitz L-15 Medium, McCoy's 5A Medium, Medium M199 (M199E—with Earle's sale base), Medium M199 (M199H—with Hank's salt base), Minimum Essential Medium Eagle (MEM-E—with Earle's salt base), Minimum Essential Medium Eagle (MEM-H—with Hank's salt base) and Minimum Essential Medium Eagle (MEM-NAA with non-essential amino acids), among numerous others, including medium 199, CMRL 1415, CMRL 1969, CMRL 1066, NCTC 135, MB 75261, MAB 8713, DM 145, Williams' G, Neuman & Tytell, Higuchi, MCDB 301, MCDB 202, MCDB 501, MCDB 401, MCDB 411, MDBC 153, and mixtures thereof in any proportions. In certain embodiments, DMEM is used. These and other useful media are available from GIBCO, Grand Island, N.Y., USA and Biological Industries, Bet HaEmek, Israel, among others.

In some embodiments, the medium may be supplemented with additional substances. Non-limiting examples of such substances are serum, which is, in some embodiments, fetal serum of cows or other species, which is, in some embodiments, 5-15% of the medium volume. In certain embodiments, the medium contains 1-5%, 2-5%, 3-5%, 1-10%, 2-10%, 3-10%, 4-15%, 5-14%, 6-14%, 6-13%, 7-13%, 8-12%, 8-13%, 9-12%, 9-11%, or 9.5%-10.5% serum, which may be FBS, or in other embodiments another animal serum.

Alternatively or in addition, the medium may be supplemented by growth factors, vitamins (e.g., ascorbic acid), cytokines, salts (e.g., B-glycerophosphate), steroids (e.g., dexamethasone) and hormones e.g., growth hormone, erythropoietin, thrombopoietin, interleukin 3, interleukin 7, macrophage colony stimulating factor, c-kit ligand/stem cell factor, osteoprotegerin ligand, insulin, insulin-like growth factor, epidermal growth factor, fibroblast growth factor, nerve growth factor, ciliary neurotrophic factor, platelet-derived growth factor, and bone morphogenetic protein.

It will be appreciated that additional components may be added to the culture medium. Such components may be antibiotics, antimycotics, albumin, amino acids, and other components known to the art for the culture of cells.

The various media described herein, e.g., the 2D growth medium and the 3D growth medium, may be independently selected from each of the described embodiments relating to medium composition. In various embodiments, any medium suitable for growth of cells in a standard tissue apparatus and/or a bioreactor may be used.

It will also be appreciated that in certain embodiments, when the described ASC are intended for administration to a human subject, the cells and the culture medium (e.g., with the above-described medium additives) are substantially xeno-free, i.e., devoid of any animal contaminants e.g., mycoplasma. For example, the culture medium can be supplemented with a serum-replacement, human serum and/or synthetic or recombinantly produced factors.

### ASC and Sources Thereof

With reference to placenta-derived ASC, “placenta”, “placental tissue”, and the like, as used herein, refer to any portion of the placenta. Placenta-derived ASC may be obtained, in various embodiments, from either fetal or, in other embodiments, maternal regions of the placenta, or in other embodiments, from both regions. More specific embodiments of maternal sources are decidua regions (e.g., decidua basalis, decidua capsularis, and decidua parietalis). More specific embodiments of fetal sources are amnion and chorion, including villous chorion. In certain embodiments, tissue specimens are washed in a physiological buffer, non-limiting examples of which are phosphate-buffered saline (PBS) and Hank's buffer. In certain embodiments, the placental tissue from which ASC are harvested includes at least one of the chorionic and decidua regions of the placenta, or, in still other embodiments, both the chorionic and decidua regions of the placenta. More specific embodiments of chorionic regions are chorionic mesenchymal and chorionic trophoblastic tissue. In a non-limiting embodiment, a mixture of maternal and fetal placental cells is obtained by mincing whole placenta; or, in other embodiments, a portion thereof; or, in still other embodiments, whole placenta, apart from the amnion, chorion, and umbilical cord.

Placental cells may be obtained, in various embodiments, from a full-term or pre-term placenta. In some embodiments, the placental tissue is optionally minced, followed by enzymatic digestion. Single-cell suspensions can be made, in other embodiments, by treating the tissue with a digestive enzyme (see below) or/and physical disruption, a non-limiting example of which is mincing and flushing the tissue parts through a nylon filter or by gentle pipetting (e.g., Falcon, Becton, Dickinson, San Jose, CA) with washing medium. In some embodiments, the tissue treatment includes use of a DNAse, a non-limiting example of which is Benzonase from Merck.

Optionally, residual blood is removed from the placenta before cell harvest. This may be done by a variety of methods known to those skilled in the art, for example by perfusion. “Perfuse” or “perfusion” herein refers to pouring or passaging a fluid over or through an organ or tissue.

In certain embodiments, the placental tissue may be from any mammal, while in other embodiments, the placental tissue is human.

Placental cells may be obtained, in various embodiments, from a full-term or pre-term placenta. A convenient source of placental tissue is a post-partum placenta (e.g., less than 48 hours after birth), however, a variety of sources of placental tissue or cells may be contemplated by the skilled person. In other embodiments, the placenta is used within 24 hours (in some embodiments, while preserved in physiological buffer), 18 hours, 14 hours, 10 hours, 8 hours, within 6 hours, within 5 hours, within 4 hours, within 3 hours, within 2 hours, or within 1 hour of birth. In certain embodiments, the placenta is kept chilled prior to harvest of the cells. In other embodiments, prepartum placental tissue is used. In some embodiments, the donor is 40 years or younger, in other embodiments 35 years old or younger, while in other embodiments, the donor may be any woman of childbearing age.

Placenta-derived cells can be propagated, in some embodiments, by using a combination of 2D and 3D culturing conditions. In certain embodiments, at least 3 passages are performed under 2D conditions, prior to 3D culturing. Conditions for propagating adherent cells in 2D and 3D culture are further described hereinbelow and in the Examples section which follows.

Those skilled in the art will appreciate, in light of the present disclosure, that cells may be, in some embodiments, extracted from a placenta, for example using physical and/or enzymatic tissue disruption, followed by marker-based cell sorting, and then may be subjected to the culturing methods described herein.

Treatment of Cells with Pro-Inflammatory Cytokines

In certain embodiments of the described methods and compositions, the composition of the medium is not varied during the course of the culturing process used to expand the placental ASC that are used in the described methods and compositions and/or for producing the described CM. In other words, no attempt is made to intentionally vary the medium composition by adding or removing factors or adding fresh medium with a different composition than the previous medium. Reference to varying the composition of the medium does not include variations in medium composition that automatically occur as a result of prolonged culturing, for example due to the absorption of nutrients and the secretion of metabolites by the cells therein, as will be appreciated by those skilled in the art.

In other embodiments, the method used to expand the steps comprises 2D culturing, followed by 3D culturing. In certain embodiments, the 3D culturing method comprises the sub-steps of: (a) incubating ASC in a 3D culture apparatus in a first growth medium, wherein no inflammatory cytokines have been added to the first growth medium; and (b) subsequently incubating the ASC in a 3D culture apparatus in a second growth medium, wherein one or more pro-inflammatory cytokines have been added to the second growth medium. Those skilled in the art will appreciate, in light of the present disclosure, that the same 3D culture apparatus may be used for the incubations in the first and second growth medium by simply adding cytokines to the medium in the culture apparatus, or, in other embodiments, by removing the medium from the culture apparatus and replacing it with medium that contains cytokines. In other embodiments, a different 3D culture apparatus may be used for the incubation in the presence of cytokines, for example by moving (e.g., passaging) the cells to a different incubator, before adding the cytokine-containing medium.

Other embodiments of pro-inflammatory cytokines, and methods comprising same, are described in WO 2017/141181 to Pluristem Ltd, by Zami Aberman et al., which is incorporated by reference herein.

### Serum-Free and Serum Replacement Media

In other embodiments, the described cell populations are produced by expanding a population of placental ASC in a medium that contains less than 5% animal serum. In certain embodiments, the cell population contains at least predominantly fetal cells (referred to as a “fetal cell population”), or, in other embodiments, contains at least predominantly maternal cells (a “maternal cell population”). In certain embodiments, the aforementioned medium contains less than 4%; less than 3%; less than 2%; less than 1%; less than 0.5%; less than 0.3%; less than 0.2%; or less than 0.1% animal serum. In other embodiments, the medium does not contain animal serum. In other embodiments, the medium is a defined medium to which no serum has been added. Low-serum and serum-free media are collectively referred to as “serum-deficient medium/media”.

Those skilled in the art will appreciate that reference herein to animal serum includes serum from any species, provided that the serum stimulates expansion of the ASC population, for example human serum, bovine serum (e.g., fetal bovine serum and calf bovine serum), equine serum, goat serum, and porcine serum.

In other embodiments, the described cell populations are produced by a process comprising: a. incubating the ASC population in a first medium, wherein the first medium contains less than 5% animal serum, thereby obtaining a first expanded cell population; and b. incubating the first expanded cell population in a second medium, wherein the second medium also contains less than 5% animal serum, and wherein 1 or more activating components are added to the second medium. This second medium can also be referred to herein as an activating medium. In other embodiments, the first medium or the second medium, or in other embodiments both the first and second medium, is/are serum free. In still other embodiments, the first medium contains a first basal medium, with the addition of one or more growth factors, collective referred to as the “first expansion medium” (to which a small concentration of animal serum is optionally added); and the activating medium contains a second basal medium with the addition of one or more growth factors (the “second expansion medium”), to which activating component(s) are added. In more specific embodiments, the second expansion medium is identical to the first expansion medium; while in other embodiments, the second expansion medium differs from the first expansion medium in one or more components.

In certain embodiments, the aforementioned step of incubating the ASC population in a first medium is performed for at least 17 doublings, or in other embodiments at least 6, 8, 12, 15, or at least 18 doublings; or 12-30, 12-25, 15-30, 15-25, 16-25, 17-25, or 18-25 doublings.

In other embodiments, the ASC population is incubated in the second medium for a defined number of days, for example 4-10, 5-10, 6-10, 4-9, 4-8, 4-7, 5-9, 5-8, 5-7, 6-10, 6-9, or 6-8; or a defined number of population doublings, for example at least 3, at least 4, at least 5, at least 6, 3-10, 3-9, 3-8, 4-10, 4-9, or 4-8. The cells are then subjected to additional culturing in the second medium in a bioreactor. In some embodiments, the bioreactor culturing is performed for at least 4, at least 5, at least 6, at least 7, at least 8, at least 9, 4-10, 4-9, 4-8, 5-10, 5-9, 5-8, 6-10, 6-9, or 6-8 population doublings; or, in other embodiments, for at least 4, at least 5, at least 6, at least 7, 4-15, 4-12, 4-10, 4-9, 4-8, 4-7, 4-15, 5-12, 5-10, 5-9, 5-8, 5-7, 6-15, 6-12, 6-10, 6-9, 6-8, or 6-7 days. In certain embodiments, the bioreactor contains 3D carriers, on which the cells are cultured.

In still other embodiments, ASC are extracted from placenta into serum-containing medium. A non-limiting extraction protocol is described in Example 1 of International Patent Application WO 2016/098061, in the name of Esther Lukasiewicz Hagai et al., published on Jun. 23, 2016, which is incorporated herein by reference in its entirety. Following initial extractions, cells are, in further embodiments, expanded in SRM. For such embodiments, the nomenclature of the aforementioned steps is retained, with the first medium (serum-replacement medium or SRM) called the “first medium”, and the activating medium called the “second [or activating] medium”.

In certain embodiments, the described serum-deficient medium is supplemented with factors intended to stimulate cell expansion in the absence of serum. Such medium is referred to herein as serum-replacement medium or SRM, and its use, for example in cell culture and expansion, is known in the art, and is described, for example, in Kinzebach et al. In still other embodiments, a chemically-defined medium is utilized.

In certain embodiments, the described SRM comprises bFGF (basic fibroblast growth factor, also referred to as FGF-2), TGF-β (TGF-β, including all isotypes, for example TGFβ1, TGFβ2, and TGFβ3), or a combination thereof. In other embodiments, the SRM comprises bFGF, TGF-β, and PDGF. In still other embodiments, the SRM comprises bFGF and TGF-β, and lacks PDGF-BB. Alternatively, or in addition, insulin is also present. In still other embodiments, an additional component selected from ascorbic acid, hydrocortisone and fetuin is present; 2 components selected from ascorbic acid, hydrocortisone and fetuin are present; or ascorbic acid, hydrocortisone and fetuin are all present.

Other SFM and SRM embodiments are disclosed in international patent application publ. no. WO 2019/186471, filed on Mar. 28, 2019, in the name of Lior Raviv et al., which is incorporated herein by reference.

### Other Embodiments of Placenta-Derived Asc

In certain embodiments, the described ASC are plastic adherent under standard culture conditions, express the surface molecules CD105, CD73 and CD90, and do not express CD45, CD34, CD14 or CD11b, CD79a, CD19 and HLA-DR. As used herein, the phrase plastic adherent refers to cells that are capable of attaching to a plastic attachment substrate and expanding or proliferating on the substrate. In some embodiments, the cells are anchorage dependent, i.e., require attachment to a surface in order to proliferate grow in vitro.

In still other embodiments, the described placenta-derived ASC (which hereinafter refers to the cells used in the described methods and compositions, or, in other embodiments, cells used to produce CM, that are used in the described methods and compositions) are a mixture of fetal-derived placental ASC (also referred to herein as “fetal ASC” or “fetal cells”) and maternal-derived placental ASC (also referred to herein as “maternal ASC” or “maternal cells”) and contains predominantly maternal cells. In more specific embodiments, the mixture contains at least 80%, at least 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9% maternal cells; or contains between 90-99%, 91-99%, 92-99%, 93-99%, 94-99%, 95-99%, 96-99%, 97-99%, 98-99%, 90-99.5%, 91-99.5%, 92-99.5%, 93-99.5%, 94-99.5%, 95-99.5%, 96-99.5%, 97-99.5%, 98-99.5%, 90-99.9%, 91-99.9%, 92-99.9%, 93-99.9%, 94-99.9%, 95-99.9%, 96-99.9%, 97-99.9%, 98-99.9% % maternal cells.

In yet other embodiments, the described cells are predominantly or completely maternal cell preparations, or are predominantly or completely fetal cell preparations, each of which represents a separate embodiment. Predominantly or completely maternal cell preparations may be obtained by methods known to those skilled in the art, including the protocol detailed in Example 1 and the protocols detailed in PCT Publ. Nos. WO 2007/108003, WO 2009/037690, WO 2009/144720, WO 2010/026575, WO 2011/064669, and WO 2011/132087. The contents of each of these publications are incorporated herein by reference. Predominantly or completely fetal cell preparations may be obtained by methods known to those skilled in the art, including selecting fetal cells via their markers (e.g., a Y chromosome in the case of a male fetus), and expanding the cells. In certain embodiments, maternal cell populations are used in the described methods and compositions. In other embodiments, fetal cells are used.

In other embodiments, the described cells are a population that does not contain a detectable number of maternal cells and is thus considered entirely fetal cells. A detectable amount refers to the number of cells detectable by FACS, using markers or combinations of markers present on maternal cells but not fetal cells, as described herein. In certain embodiments, “a detectable amount” may refer to at least 0.1%, at least 0.2%, at least 0.3%, at least 0.4%, at least 0.5%, at least 0.6%, at least 0.7%, at least 0.8%, at least 0.9%, or at least 1%.

In still other embodiments, the preparation is a mixture of fetal and maternal cells and is enriched for fetal cells. In more specific embodiments, the mixture contains at least 70% fetal cells. In more specific embodiments, at least about 30%, 35%, 40%, 45%, 50%, 55%, 60%, 65%, 75%, 80%, 85%, 90%, 95%, 96%, 97%, 98%, or 99% of the cells are fetal cells. Expression of CD200, as measured by flow cytometry, using an isotype control to define negative expression, can be used as a marker of fetal cells under some conditions. In yet other embodiments, at least 60%, 65%, 70%, 75%, 80%, 85%, 90%, 92%, 95%, 96%, 97%, 98%, 99%, or 99.5% of the described cells are fetal cells.

In more specific embodiments, the mixture contains 20-80% fetal cells; 30-80% fetal cells; 40-80% fetal cells; 50-80% fetal cells; 60-80% fetal cells; 20-90% fetal cells; 30-90% fetal cells; 40-90% fetal cells; 50-90% fetal cells; 60-90% fetal cells; 20-80% maternal cells; 30-80% maternal cells; 40-80% maternal cells; 50-80% maternal cells; 60-80% maternal cells; 20-90% maternal cells; 30-90% maternal cells; 40-90% maternal cells; 50-90% maternal cells; or 60-90% maternal cells.

In certain embodiments, the described ASC are distinguishable from human mesenchymal stromal cells (MSC)—which may, e.g., be isolated from bone marrow—as defined by The Mesenchymal and Tissue Stem Cell Committee of the ISCT (Dominici et al., 2006), based on the following 3 criteria: 1. Plastic-adherence when maintained in standard culture conditions (a minimal essential medium+20% fetal bovine serum (FBS)). 2. Expression of the surface molecules CD105, CD73 and CD90, and lack of expression of CD45, CD34, CD14 or CD11b, CD79a or CD19 and HLA-DR. 3. Ability to differentiate into osteoblasts, adipocytes and chondroblasts in vitro. By contrast, the described placental ASC are, in certain embodiments, characterized by a reduced differentiation potential, as exemplified and described further herein.

### Surface Markers and Additional Characteristics of ASC

Alternatively, or additionally, the described ASC (which are used in the described methods and compositions, or to produce CM) may express a marker or a collection of markers (e.g. surface marker) characteristic of MSC or mesenchymal-like stromal cells. In some embodiments, the ASC express some or all of the following markers: CD105 (UniProtKB Accession No. P17813), CD29 (Accession No. P05556), CD44 (Accession No. P16070), CD73 (Accession No. P21589), and CD90 (Accession No. P04216). In some embodiments, the ASC do not express some or all of the following markers: CD3 (e.g., Accession Nos. P09693 [gamma chain]P04234 [delta chain], P07766 [epsilon chain], and P20963 [zeta chain]), CD4 (Accession No. P01730), CD11b (Accession No. P11215), CD14 (Accession No. P08571), CD19 (Accession No. P15391), and/or CD34 (Accession No. P28906). In more specific embodiments, the ASC also lack expression of CD5 (Accession No. P06127), CD20 (Accession No. P11836), CD45 (Accession No. P08575), CD79-alpha (Accession No. B5QTD1), CD80 (Accession No. P33681), and/or HLA-DR (e.g. Accession Nos. P04233 [gamma chain], P01903 [alpha chain], and P01911 [beta chain]). The aforementioned, non-limiting marker expression patterns were found in certain maternal placental cell populations that were expanded on 3D substrates. All UniProtKB entries mentioned in this paragraph were accessed on Jul. 7, 2014. Those skilled in the art will appreciate that the presence of complex antigens such as CD3 and HLA-DR may be detected by antibodies recognizing any of their component parts, such as, but not limited to, those described herein.

In some embodiments, the ASC possess a marker phenotype that is distinct from bone marrow-mesenchymal stem cells (BM-MSC). In certain embodiments, the ASC are positive for expression of CD10 (which occurs, in some embodiments, in both maternal and fetal ASC); are positive for expression of CD49d (which occurs, in some embodiments, at least in maternal ASC); are positive for expression of CD54 (which occurs, in some embodiments, in both maternal and fetal ASC); are bimodal, or in other embodiments positive, for expression of CD56 (which occurs, in some embodiments, in maternal ASC); and/or are negative for expression of CD106. Except where indicated otherwise, bimodal refers to a situation where a significant percentage (e.g., at least 20%) of a population of cells express a marker of interest, and a significant percentage do not express the marker.

“Positive” expression of a marker indicates a value higher than the range of the main peak of an isotype control histogram; this term is synonymous herein with characterizing a cell as “express”/“expressing” a marker. “Negative” expression of a marker indicates a value falling within the range of the main peak of an isotype control histogram; this term is synonymous herein with characterizing a cell as “not express”/“not expressing” a marker. “High” expression of a marker, and term “highly express[es]” indicates an expression level that is more than 2 standard deviations higher than the expression peak of an isotype control histogram, or a bell-shaped curve matched to said isotype control histogram.

A cell is said to express a protein or factor if the presence of protein or factor is detectable by standard methods, an example of which is a detectable signal using fluorescence-activated cell sorting (FACS), relative to an isotype control. Reference herein to “secrete”/“secreting”/“secretion” relates to a detectable secretion of the indicated factor, above background levels in standard assays. For example, 0.5×10⁶ fetal or maternal ASC can be suspended in 4 ml medium (DMEM+10% FBS+2 mM L-Glutamine), added to each well of a 6 well-plate, and cultured for 24 hrs. in a humidified incubator (5% CO₂, at 37° C.). After 24 h, DMEM is removed, and cells are cultured for an additional 24 hrs in 1 ml RPMI 1640 medium+2 mM L-Glutamine+0.5% HSA. The CM is collected from the plate, and cell debris is removed by centrifugation.

According to some embodiments, the described ASC are capable of suppressing an immune reaction in the subject. Methods of determining the immunosuppressive capability of a cell population are well known to those skilled in the art, with exemplary methods described in Example 3 of PCT Publication No. WO 2009/144720, which is incorporated herein by reference in its entirety. For example, in an exemplary, non-limiting mixed lymphocyte reaction (MLR) assay, irradiated cord blood (iCB) cells, for example human cells or cells from another species, are incubated with peripheral blood-derived mononuclear cells (PBMC, e.g., human PBMC or PBMC from another species), in the presence or absence of a cell population to be tested. PBMC cell replication, which correlates with the intensity of the immune response, can be measured by a variety of methods known in the art, for example by ³H-thymidine uptake. Reduction of the PBMC cell replication when co-incubated with test cells indicates an immunosuppressive capability. Alternatively, a similar assay can be performed with peripheral blood (PB)-derived MNC, in place of CB cells. Alternatively, or in addition, secretion of pro-inflammatory and anti-inflammatory cytokines by blood cell populations (such as CB cells or PBMC) can be measured when stimulated (for example by incubation with non-matched cells, or with a non-specific stimulant such as PHA), in the presence or absence of the ASC. In certain embodiments, for example in the case of human ASC, as provided in WO 2009/144720, when 150,000 ASC are co-incubated for 48 hours with 50,000 allogeneic PBMC, followed by a 5-hour stimulation with 1.5 mcg/ml of LPS, the amount of IL-10 secretion by the PBMC is at least 120%, at least 130%, at least 150%, at least 170%, at least 200%, or at least 300% of the amount observed with LPS stimulation in the absence of ASC.

In other embodiments, the described ASC population secretes a therapeutic moiety, which is, in some embodiments, a secreted protein. In still other embodiments, the therapeutic moiety is Vascular Endothelial Growth Factor (VEGF). Those skilled in the art will appreciate, in light of the present disclosure, that secretion levels of VEGF can be measured by methods known in the art, e.g., the described standard ELISA protocol.

Alternatively, or in addition, the described ASC population secretes between 600-2000 pg. (=300-1000 pg/ml, as exemplified herein) of VEGF per 10⁶ cells seeded, using the standard protocol. In other embodiments, the population secretes at least 400, at least 600, at least 800, between 600-1600, between 600-1400, between 600-1200, between 800-2000, between 800-1600, between 800-1400, or between 800-1200 pg. of VEGF per 10⁶ cells seeded, using the standard protocol described herein.

In yet other embodiments, the therapeutic moiety is Angiogenin. Those skilled in the art will appreciate, in light of the present disclosure, that secretion levels of Angiogenin can be measured by methods known in the art, e.g., the described standard ELISA protocol. In certain embodiments, the described ASC population secretes between 400-800 pg. (=200-400 pg/ml, as exemplified herein) of Angiogenin per 10⁶ cells seeded, using the standard protocol.

In yet other embodiments, the therapeutic moiety is Serpin E1. Those skilled in the art will appreciate, in light of the present disclosure, that secretion levels of Serpin E1 can be measured by methods known in the art, e.g., the described standard ELISA protocol. In certain embodiments, the described ASC population secretes between 30,000-60,000 pg. (=15,000-30,000 pg/ml, as exemplified herein) of Serpin E1 per 10⁶ cells seeded, using the standard protocol.

In yet other embodiments, the therapeutic moiety is MMP-1. Those skilled in the art will appreciate, in light of the present disclosure, that secretion levels of MMP-1 can be measured by methods known in the art, e.g., the described standard ELISA protocol. In certain embodiments, the described ASC population secretes between 8000-400,000 pg. (=4000-200,000 pg/ml, as exemplified herein) of MMP-1 per 10⁶ cells seeded, using the standard protocol.

In still other embodiments, the described ASC population secretes Flt-3 ligand (Fms-related tyrosine kinase 3 ligand; Uniprot Accession No. P49772), stem cell factor (SCF; Uniprot Accession No. P21583), IL-6, or combinations thereof, each of which represents a separate embodiment. Uniprot entries in this and the following 2 paragraphs were accessed on Feb. 26, 2017.

In other embodiments, the described ASC population secretes 2 or more, in other embodiments 3 or more, in other embodiments 4 or more, in other embodiments 5 or more, in other embodiments 6 or more, in other embodiments 7 or more, or in other embodiments all of the factors VEGF, Angiogenin, PDGF, Angiopoietin 1, MCP-1, IL-8, Serpin E1, and GCP2/CXCL6.

In other embodiments, the ASC secrete VEGF, Angiogenin, Angiopoietin 1, MCP-1, IL-8, and Serpin E1, which were found to be secreted by maternal cells. In still other embodiments, the ASC secrete VEGF, Angiogenin, Angiopoietin 1, MCP-1, IL-8, Serpin E1, and GCP2/CXCL6, which were found to be secreted by fetal cells.

In other embodiments, the ASC secrete a factor(s) that promotes extracellular matrix (ECM) remodeling. In certain embodiments, the ASC secrete a factor selected from TIMP1, TIMP2, MMP-1, MMP-2, and MMP-10. In other embodiments, the ASC secrete TIMP1, TIMP2, MMP-1, MMP-2, and MMP-10, which were found to be secreted by maternal cells. In still other embodiments, the ASC secrete TIMP1, TIMP2, MMP-1, and MMP-10, which were found to be secreted by fetal cells.

In general, in certain embodiments, the described ASC exhibit a spindle shape when cultured under 2D conditions, or more specifically, are spindle in shape, with a flat, polygonal morphology, and are 15-19 μM in diameter. Alternatively or in addition, at least 90% of the cells are Oct-4 minus, as assessed by FACS. In certain embodiments, further steps of purification or enrichment for ASC may be performed. Such methods include, but are not limited to, FACS using ASC marker expression. In other embodiments, the described cells have not been subject to any type of cell sorting in the process used to isolate them. Cell sorting, in this context, refers to any a procedure, whether manual, automated, etc., that selects cells on the basis of their expression of one or more markers, their lack of expression of one or more markers, or a combination thereof. Those skilled in the art will appreciate that data from one or more markers can be used individually or in combination in the sorting process.

Alternatively or in addition, the ASC (a) have a Population Doubling Level (PDL) of no more than 25; (b) stimulate ECP and/or bone marrow migration in in vitro assays (for example, as described herein); (c) secrete, in various embodiments, at least 1, at least 2, at least 3, at least 4, at least 5, at least 6, or all 7 of IL-10, VEGF, Angiogenin, Osteopontin, IL-6, IL-8, MCP-1; (d) exhibit normal karyotype; (e) exhibit expression (in various embodiments, in at least 80%, 85%, 90%, 93%, 95%, 97%, or 98% of the cells) of CD105, CD73, CD29, and CD90; (f) exhibit lack of expression (in various embodiments, in at least 90%, 93%, 95%, 97%, 98%, 99%, 99.5%, 99.7%, or 99.75% of the cells) of CD14, CD19, CD31, CD34, CD45, HLA-DR, and CD235; or any combination of 2 or more, 3 or more, 4 or more, 5, or all 6 of characteristics a-f, each of which represents a separate embodiment. Alternatively or in addition, less than 5%, less than 10%, less than 20%, less than 30%, less than 40%, less than 50%, more than 60%, more than 70%, more than 80%, more than 90%, or more than 95% of the cells express CD200. These possibilities may be independently combined with characteristics a-f and combinations thereof, each combination representing a separate embodiment.

In certain embodiments, over 90% of the ASC are positive for CD29, CD90, and CD54. In other embodiments, over 85% of the described cells are positive for CD29, CD73, CD90, and CD105. In yet other embodiments, less than 3% of the described cells are positive for CD14, CD19, CD31, CD34, CD39, CD45RA (an isotype of CD45), HLA-DR, Glycophorin A, and CD200; less than 6% of the cells are positive for GlyA; and less than 20% of the cells are positive for SSEA4. In more specific embodiments, over 90% of the described cells are positive for CD29, CD90, and CD54; and over 85% of the cells are positive for CD73 and CD105. In still other embodiments, over 90% of the described cells are positive for CD29, CD90, and CD54; over 85% of the cells are positive for CD73 and CD105; less than 6% of the cells are positive for CD14, CD19, CD31, CD34, CD39, CD45RA, HLA-DR, GlyA, CD200, and GlyA; and less than 20% of the cells are positive for SSEA4. The aforementioned, non-limiting marker expression patterns were found in certain maternal placental cell populations that were expanded on 3D substrates.

In other embodiments, each of CD29, CD73, CD90, and CD105 is expressed by more than 80% of the ASC in each of the populations; and over 90% (or in other embodiments, over 95%, or over 98%) of the cells in each population are resistant to osteogenesis, as described in WO 2016/098061, which is incorporated herein by reference. In some embodiments, differentiation into osteocytes is assessed by incubation for 17 days with a solution containing 0.1 mcM dexamethasone, 0.2 mM ascorbic acid, and 10 mM glycerol-2-phosphate, in plates coated with vitronectin and collagen (standard osteogenesis induction conditions). In yet other embodiments, each of CD34, CD39, and CD106 is expressed by less than 10% of the cells; less than 20% of the cells highly express CD56; and the cells do not differentiate into osteocytes, after incubation under the standard conditions. In other embodiments, each of CD29, CD73, CD90, and CD105 is expressed by more than 90% of the cells, each of CD34, CD39, and CD106 is expressed by less than 5% of the cells; less than 20%, 15%, or 10% of the cells highly express CD56, and/or the cells do not differentiate into osteocytes, after incubation under the standard conditions. In still other embodiments, the conditions are incubation for 26 days with a solution containing 10 mcM dexamethasone, 0.2 mM ascorbic acid, 10 mM glycerol-2-phosphate, and 10 nM Vitamin D, in plates coated with vitronectin and collagen (modified osteogenesis induction conditions). The aforementioned solutions will typically contain cell culture medium such as DMEM+10% serum or the like, as will be appreciated by those skilled in the art. In yet other embodiments, less than 20%, 15%, or 10% of the described cells highly express CD56. In various embodiments, the cell population may be less than 50%, 40%, 30%, 20%, 10%, or 5% positive for CD200. In other embodiments, the cell population is more than 50%, 60%, 70%, 80%, 90%, 95%, 97%, 98%, 99%, or 99.5% positive for CD200. In certain embodiments, greater than 50% of the cells highly express CD141, or in other embodiments SSEA4, or in other embodiments both markers. In other embodiments, the cells highly express CD141. Alternatively or in addition, greater than 50% of the cells express HLA-A2. The aforementioned, non-limiting phenotypes and marker expression patterns were found in certain fetal tissue-derived placental cell populations that were expanded on 3D substrates, as provided herein.

In other embodiments, each of CD29, CD73, CD90, and CD105 is expressed by more than 80% of each of the ASC populations; and over 90% (or in other embodiments, over 95%, or over 98%) of the cells in each population are resistant to adipogenesis, as described in WO 2016/098061, which is incorporated herein by reference. In some embodiments, differentiation into adipocytes is assessed by incubation in adipogenesis induction medium, i.e., a solution containing 1 mcM dexamethasone, 0.5 mM 3-Isobutyl-1-methylxanthine (IBMX), 10 mcg/ml insulin, and 100 mcM indomethacin, on days 1, 3, 5, 9, 11, 13, 17, 19, and 21; and replacement of the medium with adipogenesis maintenance medium, namely a solution containing 10 mcg/ml insulin, on days 7 and 15, for a total of 25 days (standard adipogenesis induction conditions). In yet other embodiments, each of CD34, CD39, and CD106 is expressed by less than 10% of the cells; less than 20% of the cells highly express CD56; and the cells do not differentiate into adipocytes, after incubation under the standard conditions. In other embodiments, each of CD29, CD73, CD90, and CD105 is expressed by more than 90% of the cells, each of CD34, CD39, and CD106 is expressed by less than 5% of the cells; less than 20%, 15%, or 10% of the cells highly express CD56; and the cells do not differentiate into adipocytes, under the standard conditions. In still other embodiments, a modified adipogenesis induction medium, containing 1 mcM dexamethasone, 0.5 mM IBMX, 10 mcg/ml insulin, and 200 mcM indomethacin is used, and the incubation is for a total of 26 days (modified adipogenic conditions). The aforementioned solutions will typically contain cell culture medium such as DMEM+10% serum or the like, as will be appreciated by those skilled in the art. In still other embodiments, over 90% of the cells in each population do not differentiate into either adipocytes or osteocytes under the aforementioned standard conditions. In yet other embodiments, over 90% of the cells in each population do not differentiate into either adipocytes or osteocytes under the modified conditions. In various embodiments, the cell population may be less than 50%, less than 40%, less than 30%, less than 20%, or less than 10%, or less than 5% positive for CD200. In other embodiments, the cell population is more than 50%, 60%, 70%, 80%, 90%, 95%, 97%, 98%, 99%, or 99.5% positive for CD200. In certain embodiments, greater than 50% of the cells highly express CD141, or in other embodiments SSEA4, or in other embodiments both markers. In other embodiments, the cells highly express CD141. Alternatively, or in addition, greater than 50% of the cells express HLA-A2. The aforementioned, non-limiting phenotypes and marker expression patterns were found in certain fetal tissue-derived placental cell populations that were expanded on 3D substrates.

In still other embodiments, the described ASC possess any other marker phenotype, other characteristic (e.g., secretion of factor(s), differentiation capability, resistance to differentiation, inhibition of T-cell proliferation, or stimulation of myoblast proliferation), or combination thereof that is mentioned and/or described in international patent application publ. no. WO 2019/239295, filed Jun. 10, 2019, to Zami Aberman et al, which is incorporated herein by reference.

In still other embodiments, the cells are allogeneic; or in other embodiments, the cells are autologous. Alternatively, or in addition, the cells are fresh or, in other embodiments, frozen (for example, cryo-preserved).

In certain embodiments, any of the aforementioned ASC populations are used in the described methods and compositions. In other embodiments, CM obtained from the cells is used in the described methods and compositions. In still other embodiments, an exosome preparation is used. Each population may be freely combined with each of the described treatments, and each combination represents a separate embodiment. Furthermore, the cells contained in the composition or utilized to generate CM or exosomes can be, in various embodiments, autologous, allogeneic, or xenogenic to the treated subject. Each type of cell may be freely combined with the therapeutic embodiments mentioned herein.

### Additional Method Characteristics for Preparation of ASC and CM Derived Therefrom

In some embodiments, the described placental ASC have been incubated in a 3D bioreactor. Each described embodiment for cell expansion may be combined with any of the described embodiments for therapeutic uses of ASC and CM derived therefrom.

In certain embodiments, the described ASC are, or have been, subject to a 3D incubation, as described further herein. In more specific embodiments, the ASC have been incubated in a 2D adherent-cell culture apparatus, prior to the step of 3D culturing.

In some embodiments, the described ASC or CM are/is harvested from a 3D bioreactor in which the ASC have been incubated. In more specific embodiments, the cells are cryopreserved after 3D culture, and then are thawed and administered therapeutically. Alternatively or in addition, after initial 2D culturing, the cells are cryopreserved and thawed, then cultured under 2D conditions, from which the ASC are isolated and subjected to 3D culturing, e.g., in a bioreactor. In still other embodiments, the cells are expanded in 2D after initial isolation, then harvested and optionally cryopreserved and thawed, after which the cells are administered therapeutically

Reference herein to “growth” of a population of cells is intended to be synonymous with expansion of a cell population. In certain embodiments, ASC (which may be, in certain embodiments, placental ASC), are expanded without substantial differentiation. In various embodiments, the described expansion is on a 2D substrate, on a 3D substrate, or a 2D substrate, followed by a 3D substrate.

The terms “two-dimensional culture” and “2D culture” refer to a culture in which the cells are exposed to conditions that are compatible with cell growth and allow the cells to grow in a monolayer. An apparatus suitable for such growth is referred to as a “2D culture apparatus”. Such apparatuses will typically have flat growth surfaces (also referred to as a “two-dimensional substrate(s)” or “2D substrate(s)”), in some embodiments comprising an adherent material, which may be flat or curved. Non-limiting examples of apparatuses for 2D culture are cell culture dishes and plates. Included in this definition are multi-layer trays, such as Cell Factory™, manufactured by Nunc™, provided that each layer supports monolayer culture. It will be appreciated that even in 2D apparatuses, cells can grow over one another when allowed to become over-confluent. This does not affect the classification of the apparatus as “two-dimensional”.

In other embodiments, 2D culturing is performed for at least 3 passages; or, in other embodiments, at least 1, 2, or 4 passages. In other embodiments, the 2D culturing is performed for 5-15 cell doublings, in other embodiments 5-14, 5-13, 5-12, 5-11, 5-10, 6-15, 6-14, 6-13, 6-12, 6-11, or 6-10 doublings.

The terms “three-dimensional culture” and “3D culture” refer to a culture in which the cells are exposed to conditions that are compatible with cell growth and allow the cells to grow in a 3D orientation relative to one another. The term “three-dimensional [or 3D] culture apparatus” refers to an apparatus for culturing cells under conditions that are compatible with cell growth and allow the cells to grow in a 3D orientation relative to one another. Such apparatuses will typically have a 3D growth surface (also referred to as a “three-dimensional substrate” or “3D substrate”), in some embodiments comprising an adherent material, which is present in the 3D culture apparatus, e.g., the bioreactor. Certain, non-limiting embodiments of 3D culturing conditions suitable for expansion of adherent stromal cells are described in PCT Application Publ. No. WO/2007/108003, which is fully incorporated herein by reference in its entirety.

In various embodiments, “an adherent material” refers to a material that is synthetic, or in other embodiments naturally occurring, or in other embodiments a combination thereof. In certain embodiments, the material is non-cytotoxic (or, in other embodiments, is biologically compatible). Alternatively, or in addition, the material is fibrous, which may be, in more specific embodiments, a woven fibrous matrix, a non-woven fibrous matrix, or any type of fibrous matrix.

In still other embodiments, the described ASC are, or have been, subject to culturing conditions (e.g., a growth substate, incubation time, bioreactor, seeding density, or harvest density) mentioned in international patent application publ. no. WO 2019/239295, filed Jun. 10, 2019, to Zami Aberman et al, which is incorporated herein by reference.

In other embodiments, the length of 3D culturing is at least 4 days; between 4-12 days; in other embodiments between 4-11 days; 4-10 days; 4-9 days; 5-9 days; 5-8 days; 6-8 days; or 5-7 days. In other embodiments, the 3D culturing is performed for 5-15 cell doublings, in other embodiments 5-14, 5-13, 5-12, 5-11, 5-10, 6-15, 6-14, 6-13, 6-12, 6-11, or 6-10 doublings.

### Bioreactors

In certain embodiments, the described methods, or certain steps thereof, for example the 3D culturing. are performed in a bioreactor. In some embodiments, the bioreactor comprises a container for holding medium and a 3D attachment (carrier) substrate disposed therein, and a control apparatus, for controlling pH, temperature, and oxygen levels and optionally other parameters. In more specific embodiments, the 3D substrate is in a packed-bed configuration. Alternatively, or in addition, the bioreactor contains ports for the inflow and outflow of fresh medium and gases.

In certain embodiments, the aforementioned bioreactor is a packed-bed bioreactor. In some embodiments, the bioreactor comprises a container for holding medium, and a control apparatus, for controlling pH, temperature, and oxygen levels and optionally other parameters. In more specific embodiments, the bioreactor also contains a 3D substrate. Alternatively, or in addition, the bioreactor contains ports for the inflow and outflow of fresh medium and gases.

In certain embodiments, the bioreactor is connected to an external medium reservoir (e.g., that is used to perfuse the bioreactor).

The term “packed-bed bioreactor” refers to a bioreactor in which the cellular growth substrate is not ordinarily lifted from the bottom of the incubation vessel in the presence of growth medium. For example, the substrate may have sufficient density to prevent being lifted and/or it may be packed by mechanical pressure to present it from being lifted. The substrate may be either a single body or multiple bodies. Typically, the substrate remains substantially in place during perfusion at the standard perfusion rate of the bioreactor. In certain embodiments, the definition does not exclude that the substrate may be lifted at unusually fast perfusion rates, for example greater than 200 rpm.

Examples of bioreactors include, but are not limited to, a continuous stirred tank bioreactor, a CelliGen® bioreactor system (New Brunswick Scientific (NBS) and a BIOFLO 310 bioreactor system (New Brunswick Scientific (NBS).

In certain embodiments, a bioreactor is capable, in certain embodiments, of expansion of cells on a 3D substrate under controlled conditions (e.g., pH, temperature and oxygen levels) and with growth medium perfusion, which in some embodiments is constant perfusion and in other embodiments is adjusted in order to maintain target levels of glucose or other components. Furthermore, the cell cultures can be directly monitored for concentrations of glucose, lactate, glutamine, glutamate and ammonium. The glucose consumption rate and the lactate formation rate of the adherent cells enable, in some embodiments, measurement of cell growth rate and determination of the harvest time.

In some embodiments, a continuous stirred tank bioreactor is used, where a culture medium is continuously fed into the bioreactor and a product is continuously drawn out, to maintain a time-constant steady state within the reactor. A stirred tank bioreactor with a fibrous bed basket is available for example from New Brunswick Scientific Co., Edison, NJ). Additional bioreactors that may be used, in some embodiments, are stationary-bed bioreactors; and air-lift bioreactors, where air is typically fed into the bottom of a central draught tube flowing up while forming bubbles, and disengaging exhaust gas at the top of the column. Additional possibilities are cell-seeding perfusion bioreactors with polyactive foams [as described in Wendt, D. et al., Biotechnol Bioeng 84: 205-214, (2003)] and radial-flow perfusion bioreactors containing tubular poly-L-lactic acid (PLLA) porous scaffolds [as described in Kitagawa et al., Biotechnology and Bioengineering 93(5): 947-954 (2006). Other bioreactors which can be used are described in U.S. Pat. Nos. 6,277,151; 6,197,575; 6,139,578; 6,132,463; 5,902,741; and 5,629,186, which are incorporated herein by reference.

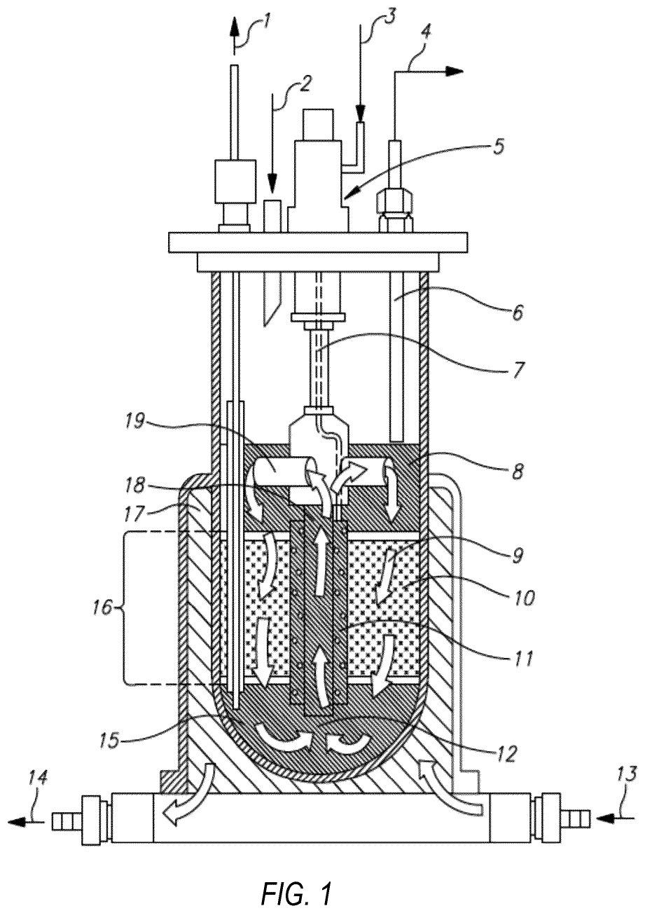

Another exemplary bioreactor, the CelliGen 310 Bioreactor, is depicted in FIG. 1. In the depicted embodiment, A Fibrous-Bed Basket (16) is loaded with polyester disks (10). In some embodiments, the vessel is filled with deionized water or isotonic buffer via an external port (1 [this port may also be used, in other embodiments, for cell harvesting]) and then optionally autoclaved. In other embodiments, following sterilization, the liquid is replaced with growth medium, which saturates the disk bed as depicted in (9). In still further embodiments, temperature, pH, dissolved oxygen concentration, etc., are set prior to inoculation. In yet further embodiments, a slow initial stirring rate is used to promote cell attachment, then the stirring rate is increased. Alternatively, or addition, perfusion is initiated by adding fresh medium via an external port (2). If desired, metabolic products may be harvested from the cell-free medium above the basket (8). In some embodiments, rotation of the impeller creates negative pressure in the draft-tube (18), which pulls cell-free effluent from a reservoir (15) through the draft tube, then through an impeller port (19), thus causing medium to circulate (12) uniformly in a continuous loop. In still further embodiments, adjustment of a tube (6) controls the liquid level; an external opening (4) of this tube is used in some embodiments for harvesting. In other embodiments, a ring sparger (not visible), is located inside the impeller aeration chamber (11), for oxygenating the medium flowing through the impeller, via gases added from an external port (3), which may be kept inside a housing (5), and a sparger line (7). Alternatively, or in addition, sparged gas confined to the remote chamber is absorbed by the nutrient medium, which washes over the immobilized cells; and/or a water jacket (17) is present, with ports for moving the jacket water in (13) and out (14).

In certain embodiments, a perfused bioreactor is used, wherein the perfusion chamber contains carriers. The carriers may be, in more specific embodiments, selected from macrocarriers, microcarriers, or either. Non-limiting examples of microcarriers that are available commercially include alginate-based (GEM, Global Cell Solutions), dextran-based (Cytodex®, GE Healthcare), collagen-based (Cultispher®, Percell Biolytica), and polystyrene-based (SoloHill Engineering) microcarriers. In certain embodiments, the microcarriers are packed inside the perfused bioreactor.

In some embodiments, the carriers in the perfused bioreactor are packed, for example forming a packed bed, which is submerged in a nutrient medium. Alternatively, or in addition, the carriers may comprise an adherent material. In other embodiments, the surface of the carriers comprises an adherent material, or the surface of the carriers is adherent. In still other embodiments, the material exhibits a chemical structure such as charged surface exposed groups, which allows cell adhesion.

Alternatively, or in addition, the carriers comprise a fibrous material, optionally an adherent, fibrous material, which may be, in further embodiments, a woven fibrous matrix, a non-woven fibrous matrix, or either. Non-limiting examples of fibrous carriers are polyester mesh-containing carriers such as New Brunswick Scientific Co.™ Fibra-Cel® disks, available commercially from Eppendorf™ AG, Germany, and including a polypropylene support; and microporous carriers such as BioNOC™ II carriers, available commercially from CESCO BioProducts (Atlanta, GA) and made of PET (polyethylene terephthalate). In certain embodiments, the referred-to fibrous matrix comprises a polyester, a polypropylene, a polyalkylene, a polyfluorochloroethylene, a polyvinyl chloride, a polystyrene, or a polysulfone. In more particular embodiments, the fibrous matrix is selected from a polyester and a polypropylene.

In other embodiments, cells are produced using a packed-bed spinner flask. In more specific embodiments, the packed bed is used in conjunction with a spinner flask and a magnetic stirrer. The spinner flask may be fitted, in some embodiments, with a packed-bed apparatus, which may be, in more specific embodiments, a fibrous matrix; a non-woven fibrous matrix; non-woven fibrous matrix comprising polyester; or a non-woven fibrous matrix comprising at least about 50% polyester. In more specific embodiments, the matrix may be disposed in a perfusion bioreactor, which is, in further embodiments, similar to the CelliGen™ bioreactor. In yet further embodiments, the bioreactor is packed with Fibra-cel® (or, in other embodiments, other carriers). The spinner is, in certain embodiments, batch fed (or in other alternative embodiments fed by perfusion), fitted with one or more sterilizing filters, and placed in a tissue culture incubator. In further embodiments, cells are seeded onto the scaffold by suspending them in medium and introducing the medium to the apparatus. In still further embodiments, the stirring speed is gradually increased, for example by starting at 40 RPM for 4 hours, then gradually increasing the speed to 120 RPM. In certain embodiments, the glucose level of the medium may be tested periodically (e.g., daily), and the perfusion speed adjusted maintain an acceptable glucose concentration, which is, in certain embodiments, between 400-700 mg\liter, between 450-650 mg\liter, between 475-625 mg\liter, between 500-600 mg\liter, or between 525-575 mg\liter. In yet other embodiments, at the end of the culture process, the carriers are removed from the packed bed, optionally washed with isotonic buffer, and processed or removed from the carriers by agitation and/or enzymatic digestion.

In certain embodiments, the bioreactor is seeded at a concentration of between 10,000-2,000,000 cells/mL of medium, or, in various embodiments 20,000-2,000,000, 30,000-1,500,000, 40,000-1,400,000, 50,000-1,300,000, 60,000-1,200,000, 70,000-1,100,000, 80,000-1,000,000, 80,000-900,000, 80,000-800,000, 80,000-700,000, 80,000-600,000, 80,000-500,000, 80,000-400,000, 90,000-300,000, 90,000-250,000, 90,000-200,000, 100,000-200,000, 110,000-1,900,000, 120,000-1,800,000, 130,000-1,700,000, or 140,000-1,600,000 cells/mL.

In still other embodiments, between 1-20×10⁶ cells per gram (gr) of carrier (substrate) are seeded, or, in various other embodiments 1.5-20×10⁶, 1.5-18×10⁶, or in other embodiments 1.8-18×10⁶, 2-18×10⁶, 3-18×10⁶, 2.5-15×10⁶, 3-15×10⁶, 3-14×10⁶, 3-12×10⁶, 3.5-12×10⁶, 3-10×10⁶, 3-9×10⁶, 4-9×10⁶, 4-8×10⁶, 4-7×10⁶, or 4.5-6.5×10⁶ cells/gr carrier.

In still other embodiments, the matrix is a bioreactor capable of both batch and perfusion modes, which is, in other embodiments, packed with Fibra-cel® carriers (or, in other embodiments, other carriers).

In other embodiments, prefabricated or rigid scaffolds are utilized. Such scaffolds require, in some embodiments, migration of cells into the scaffold, after cell seeding. In other embodiments, physically crosslinked scaffolds may be utilized, which are, in further embodiments, gels that are formed via reversible changes in pH or temperature.

In other embodiments, microencapsulation is utilized. In certain embodiments, cells are immobilized within a semi-permeable material, e.g., a membrane that allows the diffusion of nutrients, oxygen, and growth factors essential for cell growth.

In more particular embodiments, cells are removed from a 3D matrix while the matrix remains within the bioreactor. In certain embodiments, at least about 10%, 20%, or 30% of the cells are in the S and G2/M phases (collectively), at the time of harvest from the bioreactor.

In certain embodiments, the harvesting process comprises vibration or agitation, for example as described in PCT International Application Publ. No. WO 2012/140519, which is incorporated herein by reference. In certain embodiments, during harvesting, the cells are agitated at 0.7-6 Hertz, 1-3 Hertz, during, during and after, treatment with a protease, optionally also comprising a calcium chelator. In certain embodiments, the carriers containing the cells are agitated at 0.7-6 Hertz, 1-3 Hertz, while submerged in a solution or medium comprising a protease, optionally also comprising a calcium chelator.

Those skilled in the art will appreciate that a variety of isotonic buffers may be used for washing cells and similar uses. Hank's Balanced Salt Solution (HBSS; Life Technologies) is only one of many buffers that may be used.

For any preparation used in the described methods, the therapeutically effective amount or dose can be estimated initially from in vitro and cell culture assays. Often, a dose is formulated in an animal model to achieve a desired concentration or titer. Such information can be used to more accurately determine useful doses for humans.

Effective dosages and schedules for administering the compositions may be determined empirically, and making such determinations is within the skill in the art. The dosage can be adjusted by the individual physician in the event of any counterindications. A typical dosage of the ASC ranges, in some embodiments, from ˜10 million to ˜500 million cells per administration, depending on the factors mentioned above. For example, the dosage of ASC can be 10, 20, 30, 40, 50, 60, 70, 80, 90, 100, 125, 150, 175, 200, 225, 250, 275, 300, 325, 350, 375, 400, 425, 450, 475, or 500 million cells or any amount in between. It is further understood that a range of ASC can be used including from ˜100 to ˜400 million cells, from ˜150 to ˜300 million cells. Accordingly, disclosed herein are therapeutic methods, wherein the dosage of ASC administered to the subject is 10, 20, 30, 40, 50, 60, 70, 80, 90, 100, 125, 150, 175, 200, 225, 250, 275, 300, 325, 350, 375, 400, 425, 450, 475, or 500 million cells or from 150 million-300 million cells. ASC, compositions comprising ASC, and/or medicaments manufactured using said ASC can be administered in a single dose, 2 doses, 3 doses, 2-5 doses, 2-10 doses, 1-10 doses, or 1-3 doses, over a time period of 1, 2, 3-6, 6-12, 2-12, 2-20, 3-20, or 4-20 weeks; or, in other embodiments, 2 3, 4, 5, 6, 7, 8, 9, 10, 11, or 12 months; or, in other embodiments, 1.5, 2, 3, 4, 5 years, or more.

In certain embodiments, the described pharmaceutical composition contains between 100-600 million ASC, for an adult subject. In other embodiments, the pharmaceutical composition contains between 100-400 million, 100-500 million, 150-600 million, 150-500 million, 150-400 million, 200-600 million, 200-500 million, or 200-400 million ASC, for an adult subject. In still other embodiments, the composition contains between 1.5-6 million ASC per kilogram, e.g., for a pediatric subject. In yet other embodiments, e.g., for a pediatric subject, the composition contains between 1.5-5 million, 1.5-4 million, 2-5 million, 2-4 million, 3-6 million, or 3-5 million ASC per kilogram. In certain embodiments, the administration is intramuscular. The exact formulation, route of administration and dosage can be, in some embodiments, chosen by the individual physician in view of the patient's condition.

Depending on the severity and responsiveness of the condition to be treated, dosing can be of a single or, in other embodiments, a plurality of administrations, with a course of treatment lasting from 2 days to 3 weeks or, in other embodiments, from 3 weeks to 3 months, or, in other embodiments, until alleviation of the disease state is achieved.

In certain embodiments, following administration, the majority of the cells, in other embodiments more than 60%, more than 70%, more than 80%, more than 90%, more than 95%, more than 96%, more than 97%, more than 98%, or more than 99% of the cells are no longer detectable within the subject 1 month after administration.

### Formulations

In some embodiments, the described composition is an injectable composition that is manufactured by adding 1 or more excipients, e.g., stabilizers and aqueous buffers, to placental ASC or CM or exosomes thereof.

In other embodiments, the ASC or exosomes are washed to remove serum present therewith. In more specific embodiments, xenogenic serum components are reduced by at least 90%, 95%, 99%, 99.5%, 99.8%, or 99.9%; or, in other embodiments, are undetectable by standard methods, e.g., mass spectrometry.

### Additional Pharmaceutical Carriers

In certain embodiments, the described compositions comprise one or more pharmaceutically acceptable carriers. Herein, the term “pharmaceutically acceptable carrier” refers to a carrier or a diluent. In some embodiments, a pharmaceutically acceptable carrier does not cause significant irritation to a subject. In other embodiments, a pharmaceutically acceptable carrier does not abrogate the biological activity and properties of administered cells. Non-limiting examples of carriers are saline and other physiological buffers. In some embodiments, the pharmaceutical carrier is an aqueous solution of saline.

In still other embodiments, the composition comprises placental ASC in combination with an excipient selected from an osmoprotectant or cryoprotectant, an agent that protects cells from the damaging effect of freezing and ice formation. In certain embodiments, the cryoprotectant is a permeating compound, non-limiting examples of which are dimethyl sulfoxide (DMSO), glycerol, ethylene glycol, formamide, propanediol, poly-ethylene glycol, acetamide, propylene glycol, and adonitol; or may in other embodiments be a non-permeating compound, non-limiting examples of which are lactose, raffinose, sucrose, trehalose, and d-mannitol. In other embodiments, both a permeating cryoprotectant and a non-permeating cryoprotectant are present. In other embodiments, the excipient is a carrier protein, a non-limiting example of which is albumin. In still other embodiments, both an osmoprotectant and a carrier protein are present; in certain embodiments, the osmoprotectant and carrier protein may be the same compound. Alternatively, or in addition, the composition is frozen. The cells may be any embodiment of ASC mentioned herein, each of which represents a separate embodiment. In more specific embodiments, DMSO is present at a concentration of 2-5%; or, in other embodiments, 5-10%; or, in other embodiments, 2-10%, 3-5%, 4-6%; 5-7%, 6-8%, 7-9%, 8-10%. DMSO, in other embodiments, is present with a carrier protein, a non-limiting example of which is albumin, e.g., human serum albumin (HSA). In certain embodiments, HSA is present at 2-10%, 3-10%, 4-10%, 5-10%, 2-9%, 2-8%, 3-7%, 4-6%, 4.5-5.5%, or 5% (weight per volume). In still other embodiments, DMSO and HSA are both present in a saline solution (a non-limiting example of which is Plasma-Lyte® A (commercially available from Baxter).

In other embodiments, for injection, the described ASC or other active ingredients are formulated in aqueous solutions, e.g., in a physiologically compatible buffer, non-limiting examples of which are Hank's solution, Ringer's solution, and a physiological salt buffer.

### Routes

In certain embodiments, the described pharmaceutical compositions are administered intramuscularly. In other embodiments, the composition is administered systemically. Alternatively, the composition is administered locally, for example, via injection of the pharmaceutical composition directly into an exposed or affected tissue region of a patient. In other embodiments, the composition is administered intravenously (IV), subcutaneously (SC), or intraperitoneally (IP), each of which is considered a separate embodiment. In this regard, “intramuscular” administration refers to administration into the muscle tissue of a subject; “subcutaneous” to administration just below the skin; “intravenous” to administration into a vein of a subject; and “intraperitoneal” refers to administration into the peritoneum of a subject.

In various embodiments, the described ASC are administered to the subject within 3, 4, 5, 6, 8, 10, 12, or 20 days of diagnosis (or, in other embodiments, onset) of any of the herein-described conditions (each of which represents a separate embodiment.).

In various embodiments, engraftment of the described cells in the host is not required for the cells to exert the described therapeutic effects, each of which is considered a separate embodiment. In other embodiments, engraftment is required for the cells to exert the effect(s). For example, the cells may, in various embodiments, be able to exert a therapeutic effect, without themselves surviving for more than 7 days; or in other embodiments more than 4, 5, 6, 7, 8, 9, 10, or more than 14 days after administration.

Compositions including the described preparations formulated in a compatible pharmaceutical carrier may also be prepared, placed in an appropriate container, and labeled for treatment of an indicated condition.

It is clarified that each embodiment of the described ASC, CM, or exosomes may be freely combined with each embodiment relating to a therapeutic method or pharmaceutical composition.

### Subjects

In certain embodiments, the subject treated by the described methods and compositions is a human; for example, a human having a peripheral artery disease, e.g., CLI or IC. In more specific embodiments, the subject is contraindicated for surgical revascularization, or in other embodiments, for both surgical and endovascular revascularization. Those skilled in the art will appreciate that contraindications for surgical revascularization include comorbidities, for example, diabetes mellitus (DM), coronary artery disease (CAD), chronic renal failure, which may be, in certain embodiments, end-stage renal disease (ESRD) on hemodialysis. In certain embodiments, the subject has 2 or more, or in other embodiments, 3 or more, of the aforementioned comorbidities. In other embodiments, the contraindications include age >70 years, unavailability of suitable vein grafts, the absence of a landing zone for distal bypass, foot infection in the site of potential anastomosis (e.g., the dorsalis pedis or the peronea), and previous failed bypass. It will also be appreciated that impaired renal function is a contraindication for angioplasty (endovascular revascularization). In still other embodiments, the subject is a patient concurrently undergoing surgical revascularization; or, in other embodiments, who has recently (e.g., within the last month, the last 3 weeks, the last 2 weeks, or the last week) undergone surgical revascularization.

In other embodiments, the subject is an animal. In some embodiments, treated animals include domesticated animals and laboratory animals, e.g., non-mammals and mammals, for example non-human primates, rodents, pigs, dogs, and cats. In certain embodiments, the subject is administered with additional therapeutic agents or cells.

Sequential Administration of ASC from Multiple Donors

In certain embodiments, the subject is administered: (a) a first pharmaceutical composition, comprising allogeneic placental ASC from a first donor; and subsequently (b) a second pharmaceutical composition comprising allogeneic placental ASC from a second donor, wherein the second donor differs from the first donor in at least one allele group of HLA-A or HLA-B.

In certain embodiments, each of the pharmaceutical compositions contains between 100-600 million ASC, for an adult subject. In other embodiments, the pharmaceutical compositions each contain between 100-400 million, 100-500 million, 150-600 million, 150-500 million, 150-400 million, 200-600 million, 200-500 million, or 200-400 million ASC, for an adult subject. In still other embodiments, the compositions each contain between 1.5-6 million ASC per kilogram, e.g., for a pediatric subject. In yet other embodiments, e.g., for a pediatric subject, the compositions each contain between 1.5-5 million, 1.5-4 million, 2-5 million, 2-4 million, 3-6 million, or 3-5 million ASC per kilogram. In certain embodiments, the administration is intramuscular.

Reference to ASC “from” or “derived from” a donor is intended to encompass cells removed from or otherwise obtained from the donor, followed by optional steps of ex-vivo cell culture, expansion, and/or other treatments to improve the therapeutic efficacy of the cells; and/or combination with pharmaceutical excipients. Those skilled in the art will appreciate that the aforementioned optional steps will not alter the HLA genotype of the ASC, absent intentional modification of the HLA genotype (e.g., using CRISPR-mediating editing or the like). Cell populations with an intentionally modified HLA genotype are not intended to be encompassed. ASC populations that contain a mixture of cells from more than one donor are also not intended to be encompassed.

Reference to a second donor “differ/differs/differing” from a first donor in at least one allele group of HLA-A or HLA-B denotes that the DNA of the second donor comprises at least one HLA-A or HLA-B allele belonging to an allele group not represented in the alleles of the first donor. (Typically [except in the case of a homozygous first donor], the DNA of the first donor will also comprise at least one HLA-A or HLA-B allele belonging to an allele group not represented in the alleles of the second donor). Similarly, a second donor “differs from” a first donor in at least one allele supertype if the DNA of the second donor comprises at least one HLA-A or HLA-B allele belonging to a supertype not represented in the alleles of the first donor. These terms are intended to be used analogously in various contexts herein.

In other embodiments, the second donor in the described therapeutic methods and compositions differs from the first donor in at least one allele group of HLA-A. In still other embodiments, the second donor differs from the first donor in at least one allele group of HLA-B.

In yet other embodiments, the second donor differs from the first donor in at least two HLA-A allele groups of or, in other embodiments, in at least 2 HLA-B allele groups; or, in other embodiments, at least one allele group of each of HLA-A and HLA-B.

In other embodiments, the second donor differs from the first donor in at least one HLA-A allele supertype or, in other embodiments, at least one HLA-B allele supertype.

In still other embodiments, the second donor differs from the first donor in at least two allele supertypes of HLA-A or HLA-B, which may be, in more specific embodiments, an HLA-A allele supertype, an HLA-B allele supertype, or a combination thereof.

Alternatively, or in addition, the second donor differs from the first donor in at least one allele group of HLA-DR, or in other embodiments, in 2 HLA-DR allele groups.

Further embodiments of dosing regimens are described in WO 2019/239295, in the name of Zami Aberman et al., which is incorporated herein by reference.

Also disclosed herein are kits and articles of manufacture that are drawn to reagents that can be used in practicing the methods disclosed herein. The kits and articles of manufacture can include any reagent or combination of reagent discussed herein or that would be understood to be required or beneficial in the practice of the disclosed methods, including ASC. In another aspect, the kits and articles of manufacture comprise a label, instructions, and packaging material, for example for treating a disorder or therapeutic indication mentioned herein.

Except where otherwise indicated, all ranges mentioned herein are inclusive.

Unless otherwise defined, all technical and scientific terms used herein have the same meaning as commonly understood by one of ordinary skill in the art to which this invention belongs. Although methods and materials similar or equivalent to those described herein can be used in the practice or testing of the invention, suitable methods and materials are described below. In case of conflict, the patent specification, including definitions, will control. In addition, the materials, methods, and examples are illustrative only and not intended to be limiting.

Additional objects, advantages, and novel features of the invention will become apparent to one ordinarily skilled in the art upon examination of the following examples, which are not intended to be limiting. Additionally, each of the various embodiments and aspects of the invention as delineated hereinabove and as claimed in the claims section below finds experimental support in the following examples.

### EXAMPLES

Reference is now made to the following examples, which together with the above descriptions illustrate certain embodiments in a non-limiting fashion.

### Example 1: Culturing and Production of Adherent Placental Cells

Placenta-derived cell populations containing over 90% maternal tissue-derived cells were prepared as described in Example 1 of International Patent Application WO 2016/098061, which is incorporated herein by reference in its entirety. The cell expansion and harvesting process consisted of 3 stages, followed by downstream processing steps: Stage 1, the intermediate cell stock (ICS) production; Stage 2, the thawing of the ICS and initial further culture steps; and Stage 3, additional culture steps, first in tissue culture dishes, and then on Fibra-Cel® carriers in a bioreactor. All steps were performed in the presence of serum-containing medium. The downstream processing steps included harvest from flasks or bioreactor/s, cell concentration, washing, formulation, filling, and cryopreservation. The procedure included periodic testing of the growth medium for sterility and contamination, all as described in international patent application publ. no. WO 2019/239295, which is incorporated herein by reference.

### Example 2: Culture of Placental Cells in Serum-Free Medium

### Methods

The cell harvesting and expansion process was performed as described in EXAMPLE 1 above, except that all steps were performed in a serum-free medium. Bone marrow migration (BMM) assays were performed as described in WO 2019/239295.

### Results

Placental cells were extracted and expanded in serum-free (SF) medium for 3 passages. Cell characteristics of eight batches were assessed and were found to exhibit similar patterns of cell size and PDL (population doubling level since passage 1) as shown for a representative batch in Table 1. Cells also significantly enhanced hematopoiesis in a BMM assay.

TABLE 1
Characteristics of placental cells expanded in SF medium.

| | | | Total | cell | |
|---|---|---|---|---|---|
| | | | growth | size | |
| BATCH | GROUP | Passage | (days) | (μm) | PDL |
| PD200114SFM | A | 1 | 8 | 20.3 | NA |
| | | 2 | 14 | 20.9 | 3.4 |
| | | 3 | 20 | 19.7 | 7 |
| | B | 1 | 8 | 19.5 | NA |
| | | 2 | 15 | 21.5 | 3.4 |
| | | 3 | 21 | 17 | 5.1 |

| | | | |
|---|---|---|---|
| Average P 3 | 19.1 | 17.55 | 6.12 |
| % CV P 3 | 8 | 9 | 11 |

### Example 3: Osteocyte and Adipocyte Differentiation Assays

Placental adherent stromal cells (ASC) were prepared as described in Example 1. Bone marrow (BM) derived adherent cells were obtained as described in WO 2016/098061, which is incorporated herein by reference in its entirety. Osteogenesis and adipogenesis assays were performed as described in WO 2016/098061. Incubation of BM-derived adherent cells in osteogenic induction medium resulted in differentiation of over 50% of the BM cells, as demonstrated by positive alizarin red staining, while none of the placental cells exhibited signs of osteogenic differentiation. Next, a modified osteogenic medium comprising Vitamin D and higher concentrations of dexamethasone was used. Over 50% of the BM cells underwent differentiation into osteocytes, while none of the placental cells exhibited signs of osteogenic differentiation.

Adipocyte induction. Adipocyte differentiation of placenta- or BM-derived adherent cells in adipocyte induction medium resulted in differentiation of over 50% of the BM-derived cells, as demonstrated by positive oil red staining and by typical morphological changes (e.g., accumulation of oil droplets in the cytoplasm). In contrast, none of the placental-derived cells differentiated into adipocytes. Next, a modified medium containing a higher indomethacin concentration was used. Over 50% of the BM-derived cells underwent differentiation into adipocytes. In contrast, none of the placental-derived cells exhibited morphological changes typical of adipocytes.

### Example 4: Further Osteocyte and Adipocyte Differentiation Assays

ASC were prepared as described in Example 2. Adipogenesis and Osteogenesis were assessed using the STEMPRO® Adipogenesis Differentiation Kit (GIBCO, Cat #A1007001) and the STEMPRO® Osteogenesis Differentiation Kit (GIBCO, Cat #A1007201), respectively.

### Results

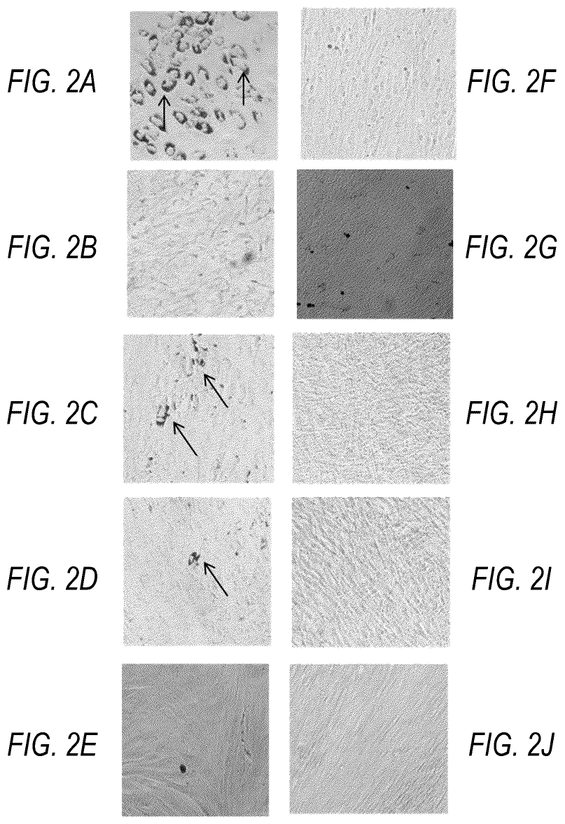

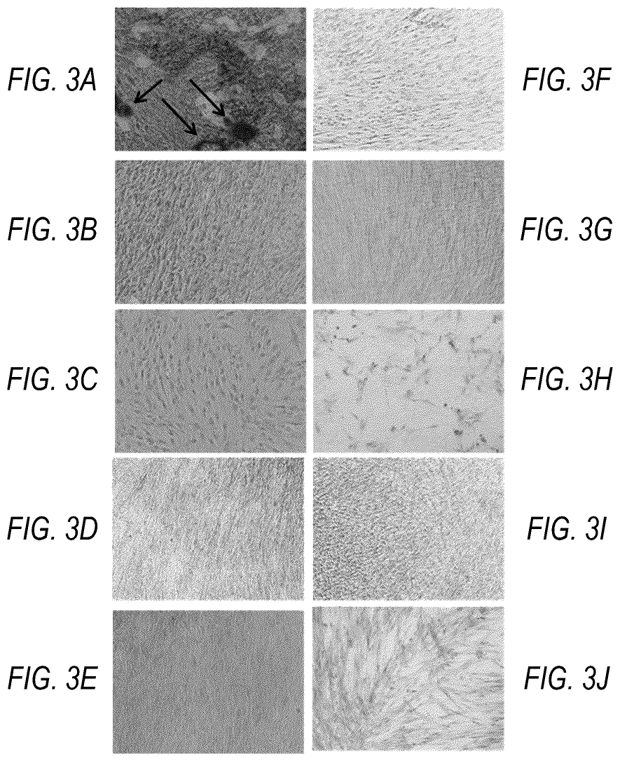

Adipogenesis and Osteogenesis of placental cells grown in serum replacement medium (SRM) (3 different batches) or in full DMEM were tested. In adipogenesis assays, BM-MSCs treated with differentiation medium stained positively with Oil Red O. By contrast, ⅔ of the SRM batches exhibited negligible staining (FIG. 2), and the other SRM batch, as well as the full DMEM-grown cells, did not exhibit any staining at all, showing that they lacked significant adipogenic potential. In osteogenesis assays, BM-MSCs treated with differentiation medium stained positively with Alizarin Red S. By contrast, none of the placental cell batches grown in SRM or full DMEM exhibited staining (FIG. 3), showing that they lacked significant osteogenic potential.

### Example 5: Clinical Study of Placental ASC for Treating Critical Limb Ischemia (CLI)

### Methods

A prospective, randomized, double-blind, multicenter, placebo-controlled, parallel-group Phase III clinical study (Norgren et al.) was performed, to evaluate the efficacy, tolerability, and safety of placental ASC for treating subjects with Critical Limb Ischemia (CLI). 213 patients exhibiting atherosclerotic CLI with minor tissue loss (Rutherford Category 5) up to the ankle level, who were unsuitable for revascularization or carried an unfavorable risk benefit for that treatment, were included. Randomization was 2:1 for drug (300 million placental ASC) vs. carrier (10% DMSO, 5% human serum albumin (HAS) and isotonic electrolyte solution [Plasma-Lyte®]), given locally, by intramuscular injection, at days 0 and 60. Screening was performed 5 weeks before randomization, followed by first drug/carrier administration 0-7 days after randomization. The primary endpoint was Amputation-Free Survival (AFS), analyzed by the Cox's proportional hazard (PH) regression model. AFS was defined as time (days) from randomization to occurrence of death or a major amputation of the index leg. The hypotheses were: H0: HR=1; H1; HR<1.

Interim analysis was performed approximately 3 years and 3 months after first randomization.

Patient numbers are summarized in Table 2 below, where:
ITT—Randomized.
mITT—Randomized and dosed.
mITT (SAP)—Randomized and dosed and had at least one visit.
Safety set—as for the mITT population but analyzed according to actual treatment received.
Legit36—all subjects in ITT who had the opportunity to complete the month 36 visit.
Legit 12—all subjects in ITT who had the opportunity to complete the month 12 visit.

TABLE 2
Summary of patient numbers.

| Description | | Number | |
|---|---|---|---|
| Screened | | 377 | |
| Randomized | | 213 | |
| Drug administration | | 205 | |
| | [symbol] | | [symbol] |
| Cohorts | Placebo | | Placental ASC |
| ITT | 70 | | 143 |
| mITT (SAP) | 69 | | 132 |
| Mitt | 69 | | 136 |
| Safety | 69 | | 136 |
| Legit 36M | 21 | | 41 |
| Legit 12M | 66 | | 130 |

Subject Disposition: Study Completion and Discontinuation are shown in Table 3:

| Parameter | Levels | Placebo | | Parameter | | Levels | |
|---|---|---|---|---|---|---|---|
| ITT | Yes | 70 | (100.0) | 143 | (100.0) | 213 | (100.0) |
| mITT | Yes | 69 | (98.6) | 136 | (95.1) | 205 | (96.2) |
| Completed study visit | Yes | 54 | (77.1) | 102 | (71.3) | 156 | (73.2) |
| month 12 | | | | | | | |
| Completed study visit | Yes | 6 | (8.6) | 7 | (4.9) | 13 | (6.1) |
| month 36 | | | | | | | |
| Reasons for early | Death* | 8 | (11.4) | 15 | (10.5) | 23 | (10.8) |
| termination | Adverse event | 2 | (2.9) | 5 | (3.5) | 7 | (3.3) |
| | Withdrawal by | 9 | (12.9) | 15 | (10.5) | 24 | (11.3) |
| | subject | | | | | | |
| | Protocol deviation | 0 | (0.0) | 1 | (0.7) | 1 | (0.5) |
| | Lost to follow-up | 0 | (0.0) | 3 | (2.1) | 3 | (1.4) |
| | Other | 49 | (70.0) | 102 | (71.3) | 151 | (70.9) |

*Of the 25 deaths occurring before randomization, 2 terminated for different reasons.

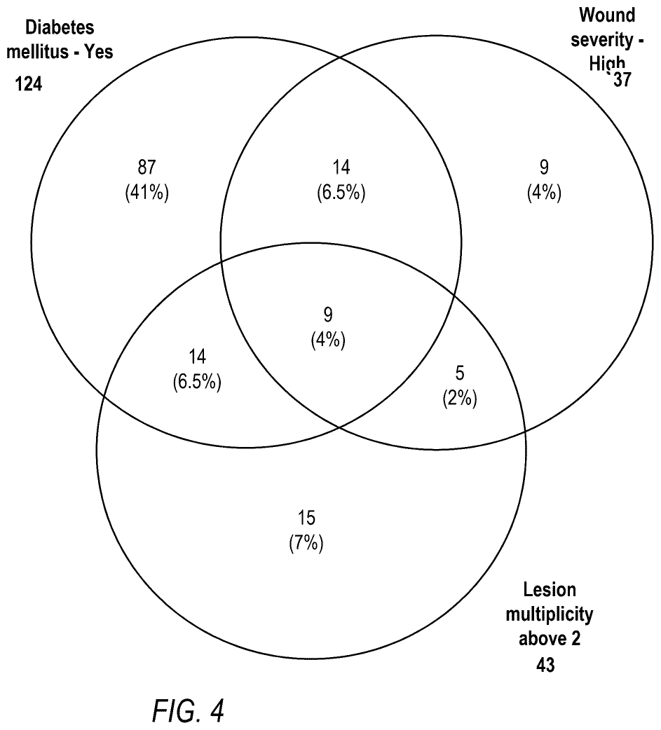

Risk factors included diabetes mellitus, severe wounds, and lesion multiplicity above 2 (FIG. 4). Table 4 sets forth patient demographics and baseline characteristics.

Demographics & Baseline Characteristics - ITT Population

| | | Placebo | | ASC | | Overall | | |
|---|---|---|---|---|---|---|---|---|
| | | (N = 70) | | (N = 143) | | (N = 213) | | |
| Parameter | Levels | n (%) | | n (%) | | n (%) | | P-value |
| SEX | Total | 70 | | 143 | | 213 | | 0.1482 |
| | Female | 21 | (30.0) | 30 | (21.0) | 51 | (23.9) | |
| | Male | 49 | (70.0) | 113 | (79.0) | 162 | (76.1) | |
| CHILDBEARING | Total | 21 | | 30 | | 51 | | |
| POTENTIAL | No | 21 | (100.0) | 30 | (100.0) | 51 | (100.0) | |
| RACE | Total | 70 | | 143 | | 213 | | 0.248 |
| | Caucasian | 66 | (94.3) | 139 | (97.2) | 205 | (96.2) | |
| | Black or | 1 | (1.4) | 1 | (0.7) | 2 | (0.9) | |
| | African- | | | | | | | |
| | American | | | | | | | |
| | Other | 3 | (4.3) | 3 | (2.1) | 6 | (2.8) | |
| ETHNIC | Total | 70 | | 143 | | 213 | | 0.5342 |
| | Hispanic | 3 | (4.3) | 8 | (5.6) | 11 | (5.2) | |
| | or Latino | | | | | | | |
| | Not | 65 | (92.9) | 133 | (93.0) | 198 | (93.0) | |
| | Hispanic | | | | | | | |
| | or Latino | | | | | | | |
| | Not | 2 | (2.9) | 1 | (0.7) | 3 | (1.4) | |
| | Reported | | | | | | | |
| | Unknown | 0 | (0.0) | 1 | (0.7) | 1 | (0.5) | |
| AGE | Ns | 70 | | 143 | | 213 | | 0.7583 |
| | MeanSD | 70.09 | (9.72) | 69.62 | (10.58) | 69.77 | (10.29) | |
| | Med | 70 | | 70 | | 70 | | |
| | MinMax | 47.00, 90.00 | | 45.00, 95.00 | | 45.00, 95.00 | | |
| HEIGHT | Ns | 70 | | 143 | | 213 | | 0.2059 |
| | MeanSD | 170.14 | (8.94) | 171.77 | (8.73) | 171.24 | (8.81) | |
| | Med | 170 | | 172 | | 172 | | |
| | MinMax | 150.00, 187.96 | | 143.00, 198.00 | | 143.00, 198.00 | | |
| WEIGHT | Ns | 70 | | 143 | | 213 | | 0.7507 |
| | MeanSD | 79.60 | (16.36) | 78.90 | (14.30) | 79.13 | (14.98) | |
| | Med | 79.5 | | 76 | | 78 | | |
| | MinMax | 42.00, 130.00 | | 40.00, 120.20 | | 40.00, 130.00 | | |
| BMI | Ns | 70 | | 143 | | 213 | | 0.3586 |
| | MeanSD | 27.27 | (4.08) | 26.70 | (4.35) | 26.89 | (4.26) | |
| | Med | 27 | | 26 | | 26 | | |
| | MinMax | 17.00, 40.00 | | 15.00, 52.00 | | 15.00, 52.00 | | |
| BSA | Ns | 70 | | 143 | | 213 | | 0.9607 |
| | MeanSD | 1.93 | (0.24) | 1.93 | (0.21) | 1.93 | (0.22) | |
| | Med | 1.9 | | 1.9 | | 1.9 | | |
| | MinMax | 1.35, 2.58 | | 1.33, 2.44 | | 1.33, 2.58 | | |
| Total area of ischemic | Ns | 66 | | 139 | | 205 | | 0.0444 |
| lesions - index leg | MeanSD | 4.85 | (4.79) | 3.57 | (3.97) | 3.98 | (4.28) | |
| | Med | 3.1 | | 1.8 | | 2.2 | | |
| | MinMax | 0.20, 19.30 | | 0.10, 17.00 | | 0.10, 19.30 | | |
| Number of lesions - | Ns | 70 | | 143 | | 213 | | 0.8168 |
| index leg | MeanSD | 1.84 | (1.29) | 1.89 | (1.36) | 1.87 | (1.33) | |
| | Med | 1 | | 1 | | 1 | | |
| | MinMax | 1.00, 7.00 | | 1.00, 10.00 | | 1.00, 10.00 | | |
| Total area of ischemic | Ns | 5 | | 4 | | 9 | | 0.4117 |
| lesions - CL leg | MeanSD | 2.16 | (2.07) | 5.33 | (7.90) | 3.57 | (5.32) | |
| | Med | 1.3 | | 2.3 | | 1.3 | | |
| | MinMax | 0.70, 5.80 | | 0.00, 16.80 | | 0.00, 16.80 | | |
| NUMBER OF | Ns | 5 | | 4 | | 9 | | 0.193 |
| LESIONS - | MeanSD | 1.40 | (0.55) | 1.00 | (0.00) | 1.22 | (0.44) | |
| CONTRALATERA | Med | 1 | | 1 | | 1 | | |
| LEG | MinMax | 1.00, 2.00 | | 1.00, 1.00 | | 1.00, 2.00 | | |
| REGION | Total | 70 | | 143 | | 213 | | 0.9438 |
| | Central/Eastern | 57 | (81.4) | 116 | (81.1) | 173 | (81.2) | |
| | Europe | | | | | | | |
| | North America | 6 | (8.6) | 14 | (9.8) | 20 | (9.4) | |
| | Western | 7 | (10.0) | 13 | (9.1) | 20 | (9.4) | |
| | Europe | | | | | | | |
| Wound severity | Total | 70 | | 143 | | 213 | | 0.7468 |
| cutoff | High Risk | 13 | (18.6) | 24 | (16.8) | 37 | (17.4) | |
| | Low Risk | 57 | (81.4) | 119 | (83.2) | 176 | (82.6) | |
| Diabetes mellitus | Total | 70 | | 143 | | 213 | | 0.9415 |
| | No | 29 | (41.4) | 60 | (42.0) | 89 | (41.8) | |
| | Yes | 41 | (58.6) | 83 | (58.0) | 124 | (58.2) | |
| Number Of Lesions | Total | 70 | | 143 | | 213 | | 0.7528 |
| Cutoff | # of Lesion <= 2 | 55 | (78.6) | 115 | (80.4) | 170 | (79.8) | |
| | # of Lesion > 2 | 15 | (21.4) | 28 | (19.6) | 43 | (20.2) | |

### Results

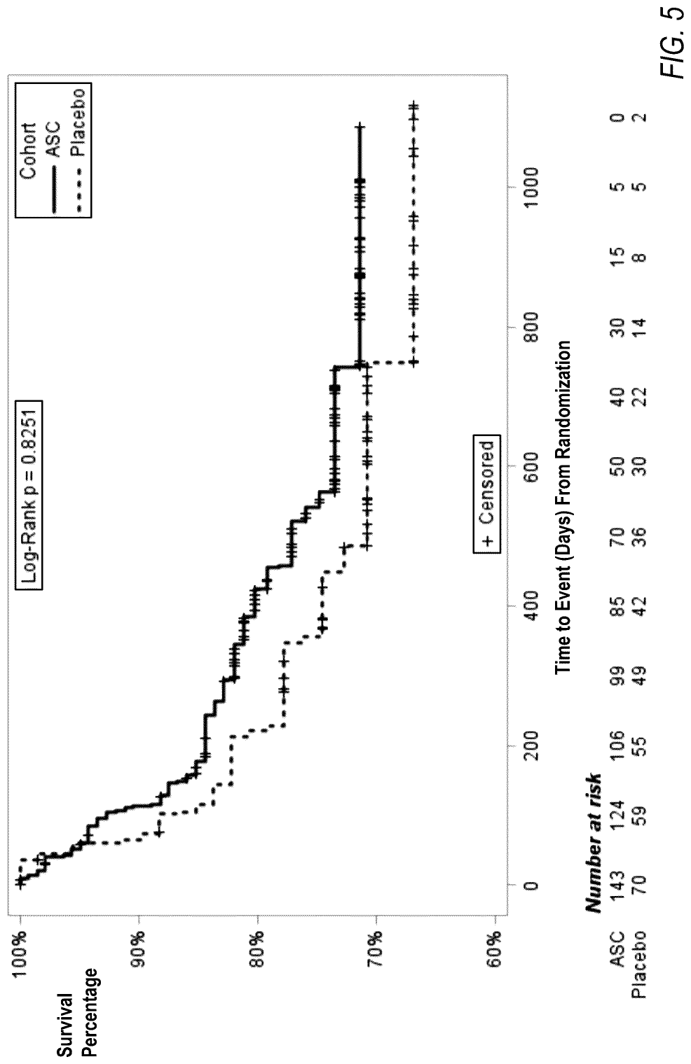

Efficacy analysis of the ITT group showed a trend of placental ASC efficacy after the second injection, but the difference did not reach statistical significance (FIGS. 5A-B and Table 5). Adverse events did not differ between the treated and control groups. Serious adverse effects in the carrier and ASC groups were reported in 60.9% and 47.1% of subjects, respectively.

Sensitivity analyses (Thabane L et al.) were performed to measure robustness of the data, by comparison to primary endpoint data of the mITT (Table 6); or measuring the hazard ratios of 2 individual components, mortality and major amputation, or the primary endpoint; or excluding covariates (Tables 7A-C, respectively); including revascularization due to worsening of the index leg; or post-hoc analysis of 1 year on the Legit 12 analysis set, all of which yielded a similar p-value of the effect of placental ASC and confirmed the robustness of the data.

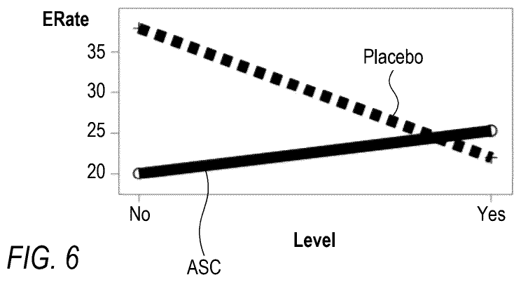

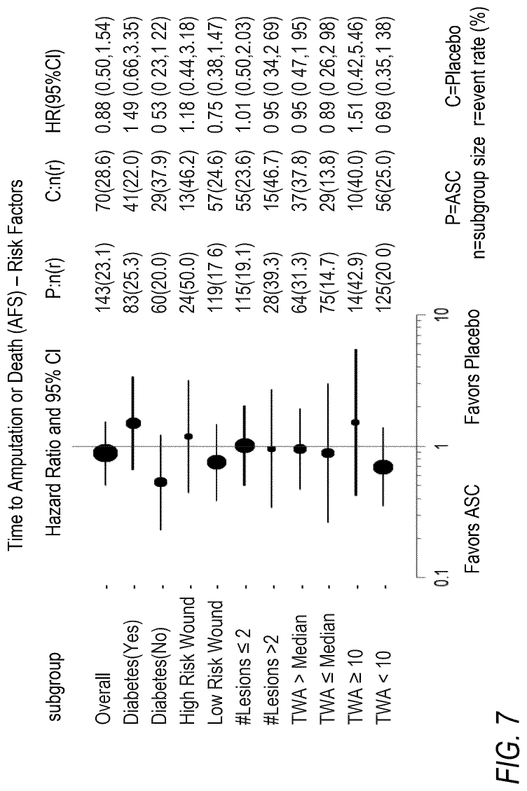

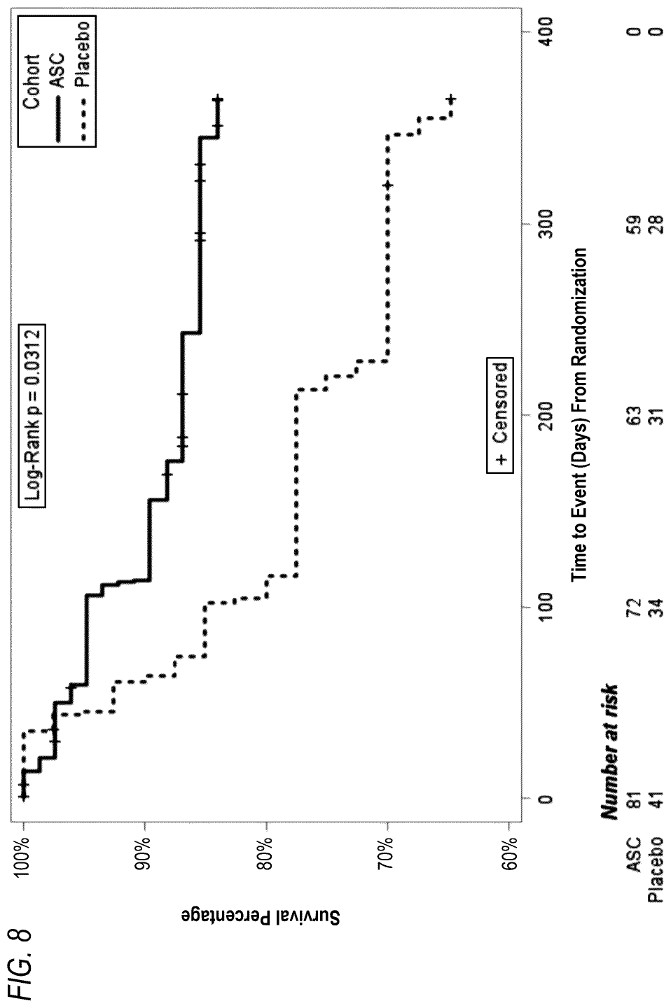

Interaction effects of cohorts showed an interaction between event rate (Erate) and diabetes status (Table 8 and FIG. 6). One- and three-year analyses showed a significant difference in the primary endpoint in subjects with Hemoglobin A1C (HbA1C)<6.4%, but not with HbA1C>6.4% (Tables 9A-C and FIG. 7; also compare FIG. 8 to FIG. 5). Similar trends were seen in the subset of patients with necrotic wounds or black toe (Tables 10A-D).

In conclusion, this experiment shows that non-diabetic subjects are particularly suited to treatment with placental ASC.

### Results Tables from Example 3

TABLE 5
Efficacy analysis of the ITT group (primary endpoint). Hazard Function is the
event rate at time t (conditioned that until time t the event has not occurred)
Primary End Point

| ITT | Placebo | | ASC | | Total | | P-value |
|---|---|---|---|---|---|---|---|
| N | 70 | | 143 | | 213 | | 0.4140 [1] |
| Number of Events n (%) | 20 | (28.6) | 33 | (23.1) | 53 | (24.9) | 0.3686 [2] |
| Number of subjects remaining at risk | 17 | (24.3) | 34 | (23.8) | | | |
| Kaplan-Meier survival probability [SE] | 66.9 | (0.066) | 71.4 | (0.045) | 69.8 | (0.038) | 0.5001 [3] |

| | |
|---|---|
| Hazard proportional assumption | 0.9298 [4] |

| | | |
|---|---|---|
| Hazard Ratio (95% CI) | 0.87 (0.50, 1.53) | 0.6329 [5] |

### Legend for Tables 5-7 and 9-10:

[1] Month 36: Logistic regression with stratification factors.
[2] Month 36: Chisq—probability difference test.
[3] Lifetest: Long rank test.
[4] PHreg: P-value COX''s model assumption (P>0.1 OK).
[5] PHref: COX's model Wald P-value.

TABLE 6
Primary endpoint analysis by mITT.
Sensitivity: Primary Endpoint by mITT

| mITT | Placebo | | ASC | | Total | | P-value |
|---|---|---|---|---|---|---|---|
| N | 69 | | 136 | | 205 | | 0.3971 |
| Number of Events n(%) | 20 | (29.0) | 31 | (22.8) | 51 | (24.9) | 0.3326 |
| Number of subjects remaining at risk | 17 | (24.6) | 34 | (25.0) | | | |
| Kaplan-Meier survival probability | 66.9 | (0.066) | 72.4 | (0.045) | 70.4 | (0.038) | 0.3764 |

| | |
|---|---|
| [SE] | |
| Hazard proportional assumption | 0.4787 |

| | | |
|---|---|---|
| Hazard Ratio (95% CI) | 0.82 (0.47, 1.45) | 0.4986 |

TABLE 7A
Sensitivity - mortality.
Sensitivity: Mortality

| ITT | Placebo | | ASC | | Total | | P-value |
|---|---|---|---|---|---|---|---|
| N | 70 | | 143 | | 213 | | 0.7297 [1] |
| Number of Events n(%) | 9 | (12.9) | 16 | (11.2) | 25 | (11.7) | 0.7223 [2] |
| Number of subjects remaining at risk | 19 | (27.1) | 34 | (23.8) | | | |
| Kaplan-Meier survival probability [SE] | 83.4 | (0.057) | 80.9 | (0.049) | 81.6 | (0.039) | 0.7922 [3] |

| | |
|---|---|
| Hazard proportional assumption | 0.3143 [4] |

| | | |
|---|---|---|
| Hazard Ratio (95% CI) | 0.97(0.42, 2.23) | 0.9355 [5] |

TABLE 7B
Sensitivity - major amputation.
Sensitivity: Major Amputation

| ITT | Placebo | | ASC | | Total | | P-value |
|---|---|---|---|---|---|---|---|
| N | 70 | | 143 | | 213 | | 0.7601 [1] |
| Number of Events n(%) | 13 | (18.6) | 24 | (16.8) | 37 | (17.4) | 0.7463 [2] |
| Number of subjects remaining at risk | 36 | (41.4) | 33 | (23.1) | | | |
| Kaplan-Meier survival probability [SE] | 78.4 | (0.054) | 77.7 | (0.044) | 77.9 | (0.034) | 0.8292 [3] |

| | |
|---|---|
| Hazard proportional assumption | 0.8758 [4] |

| | | |
|---|---|---|
| Hazard Ratio (95% CI) | 0.97(0.49, 1.92) | 0.9399 [5] |

TABLE 7C
Sensitivity analysis excluding covariates.
Sensitivity: Primary Endpoint excluding covariates

| | | | |
|---|---|---|---|
| Hazard Ratio (95% CI) | 0.82 (0.47, 1.44) | P-value | 0.5009 |

TABLE 8
Sensitivity analyses of treatment interaction.

| Main effect of cohort and interaction (Type III) | P-value | |
|---|---|---|
| Interaction | Cohort | Interaction |
| Cohort* Region {4} * | 0.3439 | 0.6939 |
| Cohort* Wound Risk {5} | 0.3837 | 0.4183 |
| Cohort* Multiplicity of wounds {5} | 0.7114 | 0.9073 |
| Cohort* Diabetes mellitus {5} | 0.4076 | 0.0758 |
| Cohort* US {6} * | 0.9506 | 0.8194 |
| Cohort* sex {7} | 0.8537 | 0.8692 |

* Group size too small

TABLE 9A
One-year primary endpoint data for subjects with HbA1C ≤6.4%.
Primary End Point - 1 Year HbA1C <=6.4%

| ITT | Placebo | | ASC | | Total | | P-value |
|---|---|---|---|---|---|---|---|
| N | 41 | | 81 | | 122 | | 0.0186 [1] |
| Number of Events n(%) | 14 | (34.1) | 12 | (14.8) | 26 | (21.3) | 0.0138 [2] |
| Number of subjects remaining at risk | 25 | (61.0) | 56 | (69.1) | | | |
| Kaplan-Meier survival probability | 64.9 | (0.076) | 83.9 | (0.043) | 77.1 | (0.040) | 0.0220 [3] |

| | |
|---|---|
| [SE] | |
| Hazard proportional assumption | 0.5125 [4] |

| | | |
|---|---|---|
| Hazard Ratio (95% CI) | 0.42 (0.20,0.92) | 0.0307 [5] |

TABLE 9B
One-year primary endpoint data for subjects with HbA1C >6.4%
Primary End Point - 1 Year HbA1C >6.4%

| ITT | Placebo | | ASC | | Total | | P-value |
|---|---|---|---|---|---|---|---|
| N | 29 | | 62 | | 91 | | 0.1323 [1] |
| Number of Events n(%) | 3 | (10.3) | 13 | (21.0) | 16 | (17.6) | 0.2184 [2] |
| Number of subjects remaining at risk | 24 | (82.8) | 41 | (66.1) | | | |
| Kaplan-Meier survival probability | 89.1 | (0.059) | 77.2 | (0.056) | 81.0 | (0.043) | 0.2153 [3] |

| | |
|---|---|
| [SE] | |
| Hazard proportional assumption | 0.8501 [4] |

| | | |
|---|---|---|
| Hazard Ratio (95% CI) | 3.15 (0.76, 12.94) | 0.1122 [5] |

TABLE 9C
Three-year primary endpoint data for subjects with HbA1C ≤6.4%.
Primary End Point - 3 Years HbA1C <=6.4%

| ITT | Placebo | | ASC | | Total | | P-value |
|---|---|---|---|---|---|---|---|
| N | 41 | | 81 | | 122 | | 0.0723 [1] |
| Number of Events n(%) | 15 | (36.6) | 16 | (21.0) | 32 | (26.2) | 0.0643 [2] |
| Number of subjects remaining at risk | 20 | (48.8) | 18 | (22.2) | | | |
| Kaplan-Meier survival probability | 61.8 | (0.078) | 72.2 | (0.064) | 68.7 | (0.049) | 0.0905 [3] |

| | |
|---|---|
| [SE] | |
| Hazard proportional assumption | 0.5357 [4] |

| | | |
|---|---|---|
| Hazard Ratio (95% CI) | 0.57 (0.28, 1.14) | 0.1127 [5] |

TABLE 10A
Time to death (one-year analysis) in subjects with black toe or necrosis

| ITT | Placebo | | ASC | | Total | | P-value |
|---|---|---|---|---|---|---|---|
| N | 25 | | 64 | | 89 | | 0.2704 [1] |
| Number of Events n(%) | 5 | (20.0) | 6 | (9.4) | 11 | (12.4) | 0.1711 [2] |
| Number of subjects remaining at risk | 17 | (68.0) | 45 | (70.3) | | | |
| Kaplan-Meier survival probability | 77.3 | (0.089) | 89.3 | (0.041) | 86.0 | (0.039) | 0.1705 [3] |

| | |
|---|---|
| [SE] | |
| Hazard proportional assumption | 0.6347 [4] |

| | | |
|---|---|---|
| Hazard Ratio (95% CI) | 0.57 (0.17, 1.95) | 0.3745 [5] |

TABLE 10B
Time to amputation (one-year analysis) in subjects with black toe or necrosis.

| ITT | Placebo | | ASC | | Total | | P-value |
|---|---|---|---|---|---|---|---|
| N | 25 | | 64 | | 89 | | 0.0323 [1] |
| Number of Events n(%) | 8 | (32.0) | 11 | (17.2) | 19 | (21.3) | 0.0357 [2] |
| Number of subjects remaining at risk | 11 | (44.0) | 40 | (62.5) | | | |
| Kaplan-Meier survival probability | 58.1 | (0.113) | 79.0 | (0.056) | 73.4 | (0.052) | 0.0724 [3] |

| | |
|---|---|
| [SE] | |
| Hazard proportional assumption | 0.5745 [4] |

| | | |
|---|---|---|
| Hazard Ratio (95% CI) | 0.33 (0.12, 0.91) | 0.0319 [5] |

TABLE 10C
Time to primary endpoint (one-year analysis) in subjects
with black toe or necrosis and HbA1C ≤6.4%.

| ITT | Placebo | | ASC | | Total | | P-value |
|---|---|---|---|---|---|---|---|
| N | 17 | | 36 | | 53 | | 0.0342 [1] |
| Number of Events n(%) | 9 | (52.9) | 8 | (22.2) | 17 | (32.1) | 0.0253 [2] |
| Number of subjects remaining at risk | 7 | (41.2) | 24 | (66.7) | | | |
| Kaplan-Meier survival probability | 43.9 | (0.124) | 75.3 | (0.076) | 64.8 | (0.069) | 0.0206 [3] |

| | |
|---|---|
| [SE] | |
| Hazard proportional assumption | 0.8987 [4] |

| | | |
|---|---|---|
| Hazard Ratio (95% CI) | 0.30 (0.11, 0.81) | 0.0170 [5] |

TABLE 10D
Time to primary endpoint (one-year analysis) in subjects
with black toe or necrosis and HbA1C >6.4%.

| ITT | Placebo | | ASC | | Total | | P-value |
|---|---|---|---|---|---|---|---|
| N | 8 | | 28 | | 36 | | 0.7886 [1] |
| Number of Events n(%) | 3 | (37.5) | 8 | (28.6) | 11 | (30.6) | 0.6287 [2] |
| Number of subjects remaining at risk | 4 | (50.0) | 17 | (60.7) | | | |
| Kaplan-Meier survival probability [SE] | 57.1 | (0.187) | 69.0 | (0.092) | 66.5 | (0.083) | 0.6002 [3] |

| | |
|---|---|
| Hazard proportional assumption | 0.4967 [4] |

| | | |
|---|---|---|
| Hazard Ratio (95% CI) | 0.91 (0.15, 5.42) | 0.9211 [5] |

### Example 6: Effect of Glucose Levels on Placental Asc

### Methods

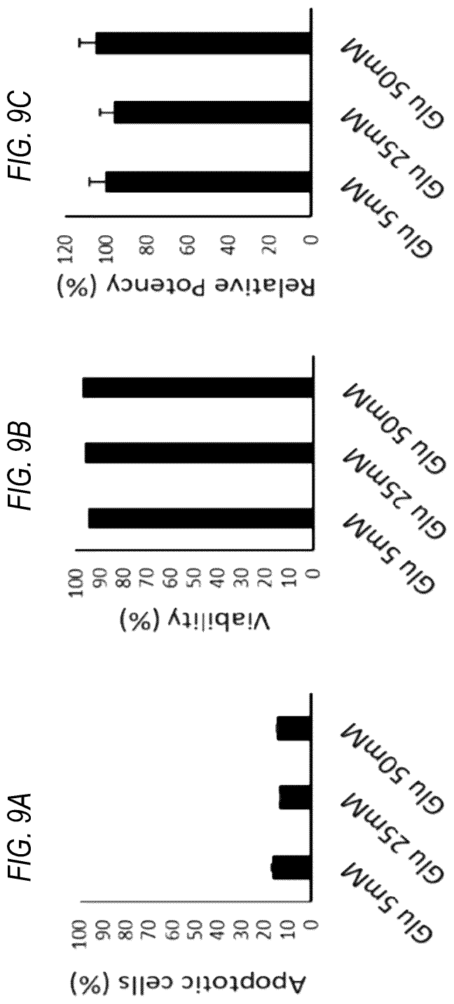

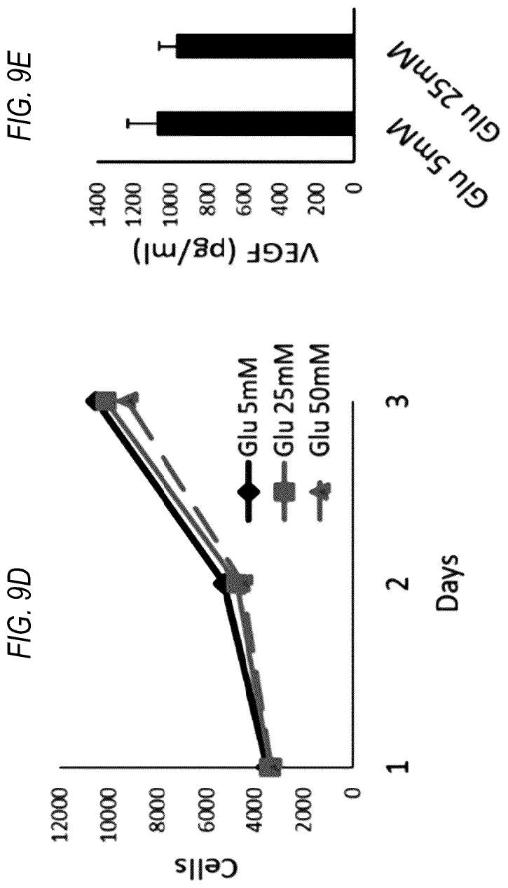

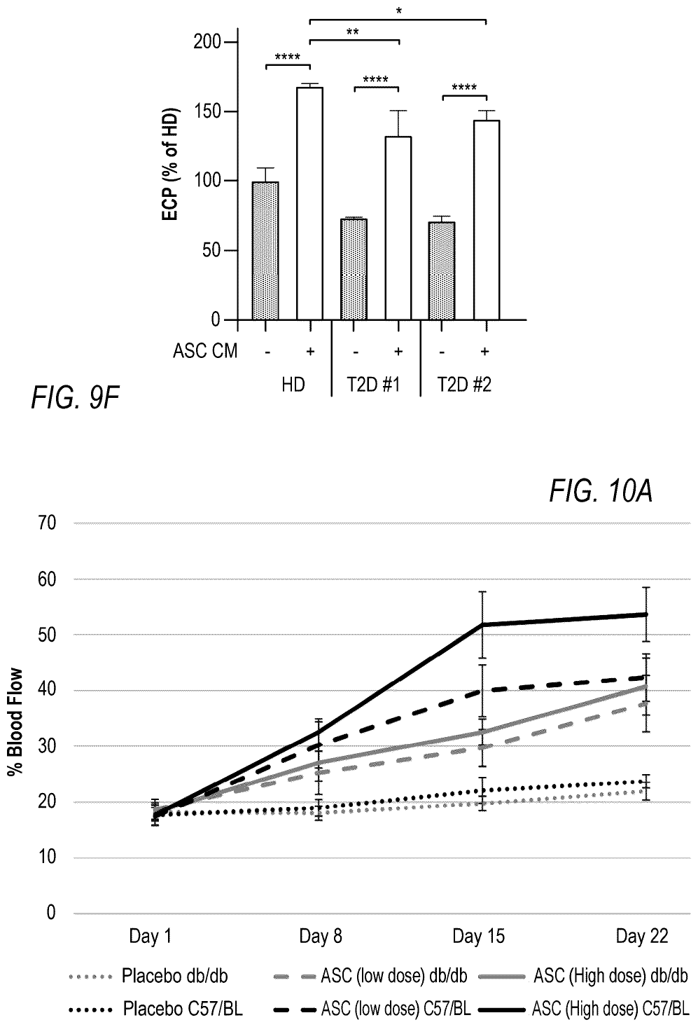

ASC were grown with the indicated glucose concentrations for 7 days and then subjected to apoptosis assay by AnnexinV staining using flow cytometry analysis (FIG. 9A), viability measurement using Vi-cell Viability Analyzer (B), cell adhesion assay (C), population doubling time assay (D) and VEGF secretion assay using an ELISA standard protocol (E). ASC-CM was added to human umbilical vein endothelial cell (HUVEC) cultures with the indicated glucose concentrations. After 96 hours, HUVEC in the wells were quantified using AlamarBlue™ (mitochondrial-activated fluorescent dye). Endothelial proliferation was normalized to growth in endothelial basal medium (EBM-2) used as a negative control.

### Results

In vitro experiments were performed to test the effect of high glucose levels on the level of apoptosis, viability, adhesion, and proliferative ability (FIG. 9A-D, respectively), and ability to secrete VEGF (FIG. 9E) of placental ASC. In all assays, ASC viability and functionality remained intact under hyperglycemic conditions. In addition, Conditioned medium (CM) of placental ASC was added to human aortic endothelial cells (HAoEC) from healthy donor (HD) and type 2 diabetic (T2D) patients (PromoCell), cultured in high glucose concentration (25 mM). After 96 hours, the quantity of HAoEC in the wells was quantified using AlamarBlue (mitochondrial-activated fluorescent dye). Endothelial proliferation was normalized to growth levels of the healthy donor. The secretions of ASC that were exposed to high glucose levels for 7 days supported endothelial cell proliferation to the same extent as ASC grown in normoglycemic conditions (FIG. 9F).

### Example 7: Effect of Placental Asc on Hind-Limb Ischemia (Hli) in Diabetic and Non-Diabetic Mice

This Example was undertaken to evaluate the effect of the placental ASC at two doses in db-db diabetic mice compared to C57/B1 normo-glycemic mice, to examine their ability to create new blood vessels and improve blood flow restoration in the treated ischemic limb of the mouse HLI model, one, two and three weeks after surgery.

### Methods

A total of 68 male mice (33 C57/BL and 35 db/db mice, obtained from Envigo RMS (Israel) Ltd or Jackson) were subjected to resection of the femoral artery (Goto et al., 2006) to induce hind-limb ischemia. Under anesthesia, the femoral artery was ligated proximally just after the distal part of the iliac artery and distally after its bifurcation with profound femoral artery with a thread, transected, and excised between the two ligatures.

The mice were divided into six groups of 11 or 12 mice per group. One day after surgery, 1×10⁴ or 1×10⁶ (total dose per mouse) placental ASC or Plasma-Lyte® (vehicle; negative control) was injected intramuscularly (IM) at the proximal and the distal sides of the surgical wound, in a volume of 25 μl at each site, total 50 μl per animal. Injection sites were tattooed to facilitate identification.

Doppler Blood flow measurements and blood sugar measurements were performed before surgery, and on Days: 1, 8, 15, and 22 (one, two, and three, weeks post-surgery).

Blood Flow Measurement Procedure: Blood flow in both legs for each mouse was measured with the LSCI system before and after surgery for inclusion criteria (only animals in which blood flow was reduced at least 30% compared to the uninjured leg were included). Animals were stratified to groups in balanced fashion according to the blood-flow results. Blood flow measurements were expressed as the ratio of the flow in the ischemic limb to that in the normal left limb.

### Histology Evaluation:

Organ/Tissue Collection and Fixation: Samples of quadriceps muscles from 12 mice were harvested (from the db/db vehicle group (termed 1M), the db/db group receiving the high dose of placental ASCs (termed 3M), the C57/B1 vehicle group (termed 4M), and the C57/B1 group receiving the high dose of placental ASCs (termed 6M)—three mice from each). Tissues were fixed in 2.5% formaldehyde and kept in fixative for a minimum of 48 hours. The tissues were trimmed, put in embedding cassettes, and processed routinely for paraffin embedding. One cassette was prepared per animal.

Slide Preparation: Paraffin sections, 4 microns thick, were cut, put on glass slides, and stained with Hematoxylin & Eosin (H&E) for general histology. Immunofluorescence (IF) staining was performed for the detection of FITC-Dextran and CD34 as markers for endothelial cells/angiogenesis.

Immunofluorescence staining protocol: FITC-DEX was injected to the animals before sacrifice. CD-34 IF staining was performed, using the Leica Bond max system (Leica Biosystems Newcastle Ltd, UK). Sections were dewaxed and pretreated with epitope-retrieval solution for 5 minutes at pH=9 (ER2, Leica Biosystems Newcastle Ltd, UK) followed by 60 minutes incubation with anti-CD34 primary antibody (Abeam ab81289, 1:100). Detection was performed, using a Cy3 Donkey Anti-Rabbit IgG secondary antibody (Jackson ImmunoResearch 711-165-152, 1:50). Nuclear staining was obtained using Hoechst 33342 (Thermo fisher cat. 62249) for five minutes.

Light and immunofluorescence Microscopy Photography: H&E—Pictures were taken using Olympus microscope (BX60, serial NO. 7D04032) equipped with microscope's Camera (Olympus DP73, serial NO. OH05504) at objective magnifications of X10. IF—The immunofluorescence-stained sections were examined by fluorescence microscope (E600, Nikon, Tokyo, Japan) equipped with Plan Fluor objective connected to a CCD camera (DMX1200F, Nikon). Digital images of 0.13 mm² (cross sections) were captured.

Digital morphometry of angiogenesis (CD34 IHC): Analysis was performed, using the Image Pro V10 (Media Cybernetics Rockville MD) software. Results are given for the ratios between the number of CD34 positive fluorescence-stained cells and the number of FITC-DEX positive blood vessels in the same section. Images were assembled using Adobe Photoshop program.

### Termination

All animals were sacrificed at day 22 (or 29).

### Results

In vivo experiments were performed to test the effect of diabetes mellitus on the efficacy of placental ASC in a mouse hind-limb ischemia model. IM administration of the placental ASC into the ischemic limb revealed an improvement in blood flow, as measured via the LASER Speckle Contrast Imaging system. The treatments in DB/DB mice restored blood perfusion with statistical significance from Day 8 up to Day 22 in the groups treated with the low dose and with the high dose of the placental ASC (also referred to herein as PLX-PAD, namely placental ASC for peripheral artery disease) compared to the Vehicle control group. In C57/B1 mice treatment restored blood perfusion with statistical significance from Day 8 up to day 22 in the groups treated with the low dose and with the high dose of the placental ASC compared to the Vehicle control group. On Days 15 and 22 statistically significant improvement in blood perfusion was seen also in C57/B1 mice between the groups treated with the low dose and with the high dose of the placental ASC. Similar atrophy and inflammation processes found in all the groups suggested similar surgical HLI damage.

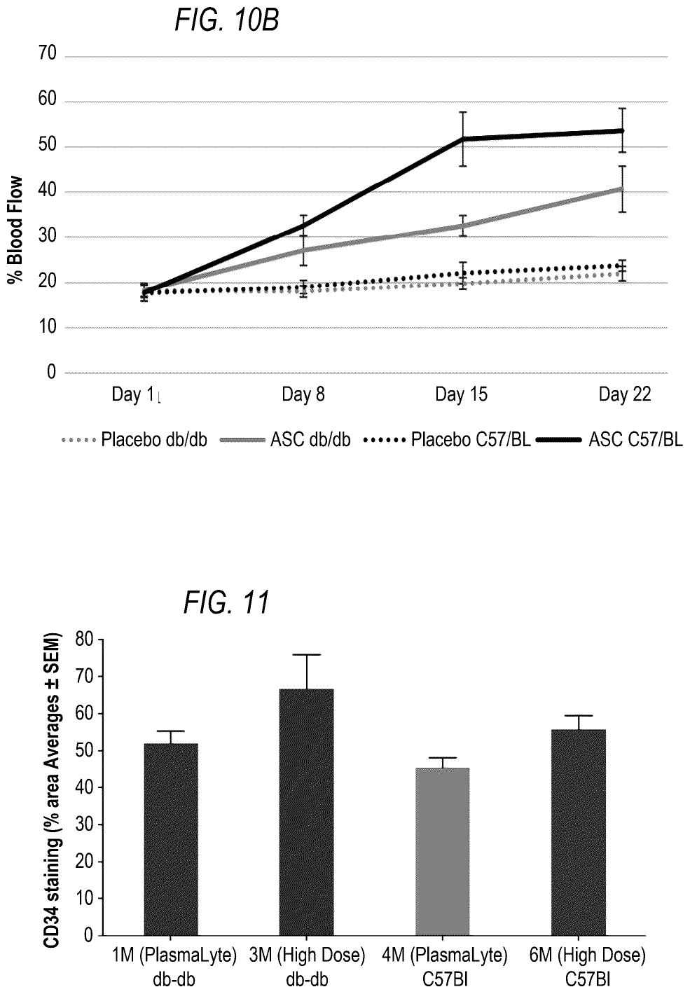

While the treatment was effective in both C57/B1 and in DB/DB, a better improvement was observed in non-diabetic mice, both in blood flow rehabilitation and in limb function (FIG. 10A). Significance of the data is more readily visualized in FIG. 10B, which shows only the carrier and high-dose groups.

The histology evaluation, using CD-34 and FITC Dextran immunostaining confirmed the above conclusion. H&E: The lesions in both muscles were mild, characterized by focal atrophy, observed only in a small number of muscle fibers, and by minor to mild diffuse inflammation, composed mainly of lymphocytes. Satellite cells were rarely observed. Slight differences were found between the muscle pathology of the Quadriceps and the Gastrocnemius muscles. The gastrocnemius muscles showed slightly more atrophy. Atrophy: All groups had mild atrophy except for one animal (in the C57/B1 group receiving the high dose of placental ASCs) that showed only minor atrophy in the Quadriceps muscle. Inflammation: All groups had the same inflammation level in both muscles. CD34/FITC ratio: The ratio was calculated over the number of positively stained blood vessels. The number of positive CD34 signals was divided by the number of Dextran positive functional vessels per picture. The ratio was higher in the db/db and C57/B1 groups which received the high dose of placental ASCs (˜1.6) than in the db/db and C57/B1 groups receiving the vehicle alone (˜1.1). These results suggest that angiogenesis was seen more in the treated placental ASC treated groups where more developing blood vessels were observed.

Morphometric results for CD34 staining are depicted in FIG. 11.

These findings further corroborate the enhanced effect of placental ASC against peripheral ischemia in non-diabetic subjects.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace alternatives, modifications and variations that fall within the spirit and broad scope of the claims and description. All publications, patents and patent applications and GenBank Accession numbers mentioned in this specification are herein incorporated in their entirety by reference into the specification, to the same extent as if each individual publication, patent or patent application or GenBank Accession number was specifically and individually indicated to be incorporated herein by reference. In addition, citation or identification of any reference in this application shall not be construed as an admission that such reference is available as prior art to the invention.

### REFERENCES

Alabi et al., Quality-of-life assessment as an outcomes measure in critical limb ischemia. J Vase Surg. 2017; 65(2): 571-578.
Bergman M et al., Review of methods for detecting glycemic disorders. Diabetes Res Clin Pract. 2020 July; 165:108233.
Booth R A et al., Ethnic dependent differences in diagnostic accuracy of glycated hemoglobin (HbA1c) in Canadian adults. Diabetes research and clinical practice. 2018; 136:143-9.
Davidson and Schriger. Effect of age and race/ethnicity on HbA1c levels in people without known diabetes mellitus: implications for the diagnosis of diabetes. Diabetes Res Clin Pract 2010; 87:415-21.
deVries et al., Comparison of generic and disease-specific questionnaires for the assessment of quality of life in patients with peripheral arterial disease. J Vase Surg. 2005; 41:261-268.
Dominici et al., Minimal criteria for defining multipotent mesenchymal stromal cells. The International Society for Cellular Therapy position statement. Cytotherapy. 2006; 8(4):315-7.
Goto T et al., Search for appropriate experimental methods to create stable hind-limb ischemia in mouse. Tokai J Exp Clin Med., Vol. 31, No. 3, pp. 118-122, 2006.
Hardman et al., Overview of Classification Systems in Peripheral Artery Disease. Semin Intervent Radiol. 2014 December; 31(4): 378-388.
Jaluvka F et al., Current Status of Cell-Based Therapy in Patients with Critical Limb Ischemia. Int J Mol Sci. 2020 Nov. 26; 21(23):8999.
Kinzebach S and Bieback K. Expansion of Mesenchymal Stem/Stromal cells under xenogenic-free culture conditions. Adv Biochem Eng Biotechnol. 2013; 129:33-57.
Norgren et al., PLX-PAD Cell Treatment of Critical Limb Ischaemia: Rationale and Design of the PACE Trial. Eur J Vasc Endovasc Surg. 2019 April; 57(4):538-545.
Thabane L et al. A tutorial on sensitivity analyses in clinical trials: the what, why, when and how. BMC Med Res Methodol. 2013; 13: 92.
Wheeler E, et al., Impact of common genetic determinants of Hemoglobin A1c on type 2 diabetes risk and diagnosis in ancestrally diverse populations: A transethnic genome-wide meta-analysis. PLoS Med 2017; 14 e1002383.

1. (canceled)

2. A method for treating critical limb ischemia (CLI) or increasing amputation-free survival (AFS) in a subpopulation of CLI subjects, comprising selecting a CLI subject wherein the subject has a normal glycemic status; and administering a therapeutically effective amount of a population of placental adherent stromal cells (ASC), therefore treating CLI or increasing amputation-free survival (AFS).

3. A method of treating critical limb ischemia (CLI) in a patient, the method comprising the steps of:
(a) determining whether the patient has diabetes mellitus; and
(b) if the patient does not have diabetes mellitus, then intramuscularly administering placental ASC to the patient, and
(c) if the patient does have diabetes mellitus, then administering an alternative therapy to the patient.

4. The method of claim 3 wherein said step of determining whether the patient has diabetes mellitus is performed by:
(i) obtaining or having obtained a biological sample(s) from the patient; and
(ii) performing or having performed an assay on the biological sample to measure a parameter selected from the group consisting of hemoglobin AIC glycosylation, post-prandial plasma glucose level, and fasting plasma glucose level.

5. The method of claim 4 wherein said treating CLI comprises one or more of increasing AFS, decreasing likelihood of requiring revascularization, decreasing likelihood of developing gangrene, inhibiting development of necrosis, healing wounds, decreasing likelihood of worsening of wounds, or improving perfusion of an ischemic limb.

6. A method of increasing amputation-free survival (AFS) in a subject with critical limb ischemia (CLI), comprising:
a. screening the subject for the presence or absence of diabetes mellitus;
b. administering placental adherent stromal cells (ASC) to the subject if said diabetes mellitus is determined to be absent; and
c. applying an alternative therapy to the subject if said diabetes mellitus is determined to be present, thereby increasing AFS in a subject with CLI.

7. (canceled)

8. A composition for treating critical limb ischemia (CLI), comprising placental adherent stromal cells (ASC), wherein said composition is indicated for use in a subject with normal glycemic status.

9. The composition of claim 8 wherein said treating CLI comprises one or more of increasing AFS, decreasing likelihood of requiring revascularization, decreasing likelihood of developing gangrene, inhibiting development of necrosis, healing wounds, decreasing likelihood of worsening of wounds, or improving perfusion of an ischemic limb.

10. A composition for increasing amputation-free survival (AFS) in a subject with critical limb ischemia (CLI), comprising placental adherent stromal cells (ASC), wherein said composition is indicated for use in a subject with normal glycemic status.

11. The composition of claim 8, where said composition is an injected composition.

12. The composition of claim 8, wherein said placental ASC have been incubated on a 2D substrate.

13. The composition of claim 8, wherein said placental ASC have been incubated on a 3D substrate.

14. (canceled)

15. The composition of claim 13, wherein said 3D culture substrate comprises a synthetic adherent material.

16. (canceled)

17. The composition of claim 15, wherein said synthetic adherent material is selected from the group consisting of a fibrous matrix, a polyester, a polypropylene, a polyalkylene, a polyfluorochloroethylene, a polyvinyl chloride, a polystyrene, and a polysulfone.

18. The composition of claim 13, wherein said 3D culture apparatus comprises microcarriers disposed within a bioreactor.

19. The composition of claim 8, wherein said placental ASC are allogeneic to said subject.

20. The composition of claim 8, wherein the composition is intramuscularly injected.

21. The composition of claim 8, comprising 100-600 million of said placental ASC, for an adult subject.

22. The composition of claim 8, wherein said placental ASC express a marker selected from the group consisting of CD73, CD90, CD29 and CD105.

23. The composition of claim 8, wherein said placental ASC do not express a marker selected from the group consisting of CD3, CD4, CD11b, CD14, CD19, and CD34.

24. (canceled)

25. The composition of claim 8, wherein said placental ASC do not express a marker selected from the group consisting of CD3, CD4, CD34, CD39, and CD106.

26. (canceled)

27. (canceled)
